(19) United States
(12) Patent Application Publication
(10) Pub. No.: US 2024/0347169 A1
(43) Pub. Date: Oct. 17, 2024

(54) ELECTRONIC APPARATUS AND CONTROLLING METHOD THEREOF

(71) Applicant: Samsung Electronics Co., Ltd., Suwon-si (KR)

(72) Inventors: Minjung KIM, Suwon-si (KR); Wooil KIM, Suwon-si (KR); Taehun KIM, Suwon-si (KR); Donghyun ROH, Suwon-si (KR); Jinwoo SONG, Suwon-si (KR); Jaehun LEE, Suwon-si (KR); Jungbae YI, Suwon-si (KR); Hwangrae LEE, Suwon-si (KR); Jinmook LIM, Suwon-si (KR)

(21) Appl. No.: 18/594,639

(22) Filed: Mar. 4, 2024

Related U.S. Application Data
(63) Continuation of application No. PCT/KR2024/002075, filed on Feb. 14, 2024.

(30) Foreign Application Priority Data
Apr. 14, 2023 (KR) 10-2023-0049538
Sep. 11, 2023 (KR) 10-2023-0120685

(51) Int. Cl. G16H 20/60

(52) U.S. Cl. CPC G16H 20/60

(57) ABSTRACT

An electronic apparatus and a controlling method thereof are provided. The electronic apparatus includes memory storing one or more computer programs, and one or more processors communicatively coupled to the memory, wherein the one or more computer programs include computer-executable instructions executed by the one or more processors and wherein the one or more processors configured to acquire nutritional ingredient information corresponding to food information in case of acquiring the food information, acquire candidate food probability information based on the nutritional ingredient information, and acquire final food group capacity information corresponding to the food information based on the nutritional ingredient information and the candidate food probability information.

### CROSS-REFERENCE TO RELATED APPLICATION(S)

This application is a continuation application, claiming priority under § 365 (c), of an International application No. PCT/KR2024/002075, filed on Feb. 14, 2024, which is based on and claims the benefit of a Korean patent application number 10-2023-0049538, filed on Apr. 14, 2023, in the Korean Intellectual Property Office, and of a Korean patent application number 10-2023-0120685, filed on Sep. 11, 2023, in the Korean Intellectual Property Office, the disclosure of each of which is incorporated by reference herein in its entirety.

### TECHNICAL FIELD

The disclosure relates to an electronic apparatus and a controlling method thereof. More particularly, the disclosure relates to an electronic apparatus which provides an analysis result related to food information and a controlling method thereof.

### BACKGROUND ART

A diet or a meal schedule that is suitable for a user may be provided using information on various foods consumed by the user. However, it may be difficult for the user to determine all the information on the various foods consumed at least once a day.

Even in case that the user knows food information, it may be cumbersome for the user to input all corresponding information.

Even different foods may have some nutrients in common. In addition, an international dietary guideline may be increasingly based on a food group. Therefore, a food recommended to the user may need to be a specific food group rather than a specific food.

The above information is presented as background information only to assist with an understanding of the disclosure. No determination has been made, and no assertion is made, as to whether any of the above might be applicable as prior art with regard to the disclosure.

### DISCLOSURE

### Technical Solution

Aspects of the disclosure are to address at least the above-mentioned problems and/or disadvantages and to provide at least the advantages described below. Accordingly, an aspect of the disclosure provides an electronic apparatus which classifies food groups based on food information and acquires a corresponding capacity for each food group, and a controlling method thereof.

Additional aspects will be set forth in part in the description which follows and, in part, will be apparent from the description, or may be learned by practice of the presented embodiments.

In accordance with an aspect of the disclosure, an electronic apparatus is provided. The electronic apparatus includes memory storing one or more computer programs, and one or more processors communicatively coupled to the memory, wherein the one or more computer programs include computer-executable instructions executed by the one or more processors, wherein the one or more processors may receive a user input for a food, acquire food information corresponding to the food from the received user input, acquire nutritional ingredient information of the acquired food information by inputting the acquired food information into a nutritional ingredient module, acquire candidate food probability information by inputting the nutritional ingredient information into a candidate food probability module, acquire food group probability information and preliminary food group capacity information by inputting the acquired nutritional ingredient information and the acquired candidate food probability information respectively into a food group probability module and a food group capacity module, acquire filtered food group probability information by removing a probability value less than a threshold value from the acquired food group probability information, and acquire final food group capacity information by coupling the filtered food group probability information with the preliminary food group capacity information.

The one or more processors may be configured to acquire food group threshold value information including the threshold value corresponding to each of a plurality of food groups, and acquire the filtered food group probability information by removing the probability value less than the threshold value included in the threshold value information from the acquired food group probability information for each of the plurality of food groups.

The one or more processors may be configured to acquire the preliminary food group capacity information indicating capacity information for each of a plurality of food groups by inputting the nutritional ingredient information and the candidate food probability information into the food group capacity module.

The user input for the food includes at least one of text information, image information, or audio information, indicating a specific food.

The one or more processors may be configured to store, in the memory, a user history including accumulated capacity information for each of a plurality of food groups consumed by a user during a predetermined time period, and update the user history by accumulating a capacity value for each of the plurality of food groups that is included in the final food group capacity information.

The one or more processors may be configured to acquire score information related to a user eating habit by comparing reference capacity information with the accumulated capacity information for each of the plurality of food groups that is included in the updated user history.

The score information includes at least one of a user eating habit score, a user age, or an average score of the user age.

The one or more processors may be configured to provide guide information indicating a recommended eating habit for a first food group by comparing a first capacity value of the first food group that is included in the updated user history with a first reference value corresponding to the first food group that is included in the reference capacity information.

The guide information includes at least one of information related to an insufficient food group or information related to an excessively consumed food group.

The apparatus further includes a display, wherein the one or more processors are configured to control the display to display a guide screen including the food information and the final food group capacity information.

In accordance with another aspect of the disclosure, a method of controlling an electronic apparatus is provided. The method includes receiving a user input for a food, acquiring food information corresponding to the food from the received user input, acquiring nutritional ingredient information of the acquired food information by inputting the acquired food information into a nutritional ingredient module, acquiring candidate food probability information by inputting the nutritional ingredient information into a candidate food probability module, acquiring food group probability information and preliminary food group capacity information by inputting the acquired nutritional ingredient information and the acquired candidate food probability information respectively into a food group probability module and a food group capacity module, acquiring filtered food group probability information by removing a probability value less than a threshold value from the acquired food group probability information, and acquiring final food group capacity information by coupling the filtered food group probability information with the preliminary food group capacity information.

In the acquiring of the filtered food group probability information, food group threshold value information including the threshold value corresponding to each of a plurality of food groups is acquired, and the filtered food group probability information is acquired by removing the probability value less than the threshold value included in the threshold value information from the acquired food group probability information for each of the plurality of food groups.

In the acquiring of the preliminary food group capacity information, the preliminary food group capacity information indicating capacity information for each of a plurality of food groups is acquired by inputting the nutritional ingredient information and the candidate food probability information into the food group capacity module.

The user input for the food includes at least one of text information, image information, or audio information, indicating a specific food.

The method further includes storing a user history including accumulated capacity information for each of a plurality of food groups consumed by a user during a predetermined time period, and updating the user history by accumulating a capacity value for each of the plurality of food groups that is included in the final food group capacity information.

The method further includes acquiring score information related to a user eating habit by comparing reference capacity information with the accumulated capacity information for each of the plurality of food groups that is included in the updated user history.

The score information includes at least one of a user eating habit score, a user age, or an average score of the user age.

The method further includes providing guide information indicating a recommended eating habit for a first food group by comparing a first capacity value of the first food group that is included in the updated user history with a first reference value corresponding to the first food group that is included in the reference capacity information.

The guide information includes at least one of information related to an insufficient food group or information related to an excessively consumed food group.

The method further includes displaying a guide screen including the food information and the final food group capacity information.

Other aspects, advantages, and salient features of the disclosure will become apparent to those skilled in the art from the following detailed description, which, taken in conjunction with the annexed drawings, discloses various embodiments of the disclosure.

### DESCRIPTION OF DRAWINGS

The above and other aspects, features, and advantages of certain embodiments of the disclosure will be more apparent from the following description taken in conjunction with the accompanying drawings, in which:

FIG. 1 is a view illustrating an artificial intelligence model according to an embodiment of the disclosure;

FIG. 2 is a view illustrating at least one module included in an artificial intelligence model according to an embodiment of the disclosure;

FIG. 3 is a block diagram illustrating an electronic apparatus according to an embodiment of the disclosure;

FIG. 4 is a block diagram illustrating a specific configuration of an electronic apparatus of FIG. 2 according to an embodiment of the disclosure;

FIG. 5 is a view illustrating an operation of acquiring nutritional ingredient information according to an embodiment of the disclosure;

FIG. 6 is a view illustrating an operation of acquiring probability information of a candidate food according to an embodiment of the disclosure;

FIG. 7 is a view illustrating an operation of acquiring candidate food probability information by using an artificial intelligence model according to an embodiment of the disclosure;

FIG. 8 is a view illustrating an operation of acquiring food group probability information according to an embodiment of the disclosure;

FIG. 9 is a view illustrating an operation of acquiring food group probability information by using an artificial intelligence model according to an embodiment of the disclosure;

FIG. 10 is a view illustrating an operation of acquiring preliminary food group capacity information according to an embodiment of the disclosure;

FIG. 11 is a view illustrating an operation of acquiring preliminary food group capacity information by using an artificial intelligence model according to an embodiment of the disclosure;

FIG. 12 is a view illustrating a filtering operation of probability information according to an embodiment of the disclosure;

FIG. 13 is a view illustrating an operation of acquiring final food group capacity information according to an embodiment of the disclosure;

FIG. 14 is a flowchart illustrating an operation of acquiring final food group capacity information according to an embodiment of the disclosure;

FIG. 15 is a flowchart illustrating an operation of acquiring final food group capacity information by using food group probability information and preliminary food group capacity information according to an embodiment of the disclosure;

FIG. 16 is a flowchart illustrating an operation of acquiring candidate food probability information according to an embodiment of the disclosure;

FIG. 17 is a flowchart illustrating a filtering operation of food group probability information according to an embodiment of the disclosure;

FIG. 18 is a flowchart illustrating an operation of providing a guide screen according to an embodiment of the disclosure;

FIG. 19 is a view illustrating a screen for inputting food information according to an embodiment of the disclosure;

FIG. 20 is a view illustrating a screen for inputting food information by using an image according to an embodiment of the disclosure;

FIG. 21 is a view illustrating a guide screen related to a user history according to an embodiment of the disclosure;

FIG. 22 is a view illustrating a screen related to an eating habit score according to an embodiment of the disclosure;

FIG. 23 is a view illustrating a screen related to a food group score according to an embodiment of the disclosure;

FIG. 24 is a view illustrating a screen related to an insufficient food group according to an embodiment of the disclosure;

FIG. 25 is a view illustrating a screen related to a recommended eating habit according to an embodiment of the disclosure;

FIG. 26 is a view illustrating a screen providing guide information for purchase of an insufficient food group according to an embodiment of the disclosure;

FIG. 27 is a view illustrating a screen providing restaurant information related to an insufficient food group according to an embodiment of the disclosure;

FIG. 28 is a flowchart illustrating an operation of acquiring final food group capacity information by using a server according to an embodiment of the disclosure;

FIG. 29 is a flowchart illustrating an operation of generating guide information by using a server according to an embodiment of the disclosure; and

FIG. 30 is a flowchart illustrating a controlling method of an electronic apparatus according to an embodiment of the disclosure.

Throughout the drawings, it should be noted that like reference numbers are used to depict the same or similar elements, features, and structures.

### MODE FOR INVENTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

General terms that are currently widely used are selected as terms used in embodiments of the disclosure based on their functions in the disclosure. However, these terms may be changed depending on the intention of those skilled in the art or a judicial precedent, the emergence of a new technique, and the like. In addition, in a specific case, terms arbitrarily chosen by an applicant may exist. In this case, the meanings of such terms are mentioned in corresponding descriptions of the disclosure. Therefore, the terms used in the disclosure need to be defined based on the meanings of the terms and the contents throughout the disclosure rather than simple names of the terms.

In the specification, an expression “have,” “may have,” “include,” “may include,” or the like, indicates the existence of a corresponding feature (for example, a numerical value, a function, an operation or a component, such as a part), and does not exclude the existence of an additional feature.

An expression, “at least one of A or/and B” may indicate either “A or B”, or “both of A and B.”

Expressions “first,” “second,” and the like, used in the specification may qualify various components regardless of the sequence or importance of the components. The expression is used only to distinguish one component from another component, and does not limit the corresponding component.

In case that any component (for example, a first component) is mentioned to be “(operatively or communicatively) coupled with/to” or “connected to” another component (for example, a second component), it is to be understood that any component may be directly coupled to another component or may be coupled to another component through still another component (for example, a third component).

It is to be understood that a term “include,” “formed of,” or the like used in this application specifies the existence of features, numerals, steps, operations, components, parts, or combinations thereof, which is mentioned in the specification, and does not preclude the existence or addition of one or more other features, numerals, steps, operations, components, parts, or combinations thereof.

In the disclosure, a “module” or a “˜er/˜or” may perform at least one function or operation, may be implemented by hardware or software, or may be implemented by a combination of hardware and software. In addition, a plurality of “modules” or a plurality of “˜ers/˜ors” may be integrated in at least one module to be implemented by at least one processor (not shown) except for a “module” or a “˜er/or” that needs to be implemented by specific hardware.

In the specification, such a term as a “user” may refer to a person who uses an electronic apparatus or an apparatus (for example, an artificial intelligence electronic apparatus) which uses the electronic apparatus.

Hereinafter, the embodiments of the disclosure are described with reference to the accompanying drawings.

In the disclosure, an artificial intelligence model being trained may indicate that the basic artificial intelligence model (e.g., the artificial intelligence model including any random parameter) is trained using a large number of training data based on a learning algorithm, thereby generating a predefined operation rule or the artificial intelligence model, set to perform a desired feature (or purpose). The learning may be conducted through a separate server and/or system, is not limited thereto, and may also be accomplished by an electronic apparatus 100. Examples of the learning algorithms may include supervised learning, unsupervised learning, semi-supervised learning, transfer learning, or reinforcement learning, and are not limited to these examples.

Here, each artificial intelligence model may be implemented, for example, as a convolutional neural network (CNN), a recurrent neural network (RNN), a restricted Boltzmann machine (RBM), a deep belief network (DBN), a bidirectional recurrent deep neural network (BRDNN), or a deep Q-network, and is not limited thereto.

At least one processor 120 for executing the artificial intelligence model according to an embodiment of the disclosure may be implemented through a combination of a processor and software, the processor including a general-purpose processor, such as a central processing unit (CPU), an application processor (AP) or a digital signal processor (DSP), a graphics-only processor, such as a graphics processing unit (GPU) or a vision processing unit (VPU), or a processor dedicated to an artificial intelligence, such as a neural processing unit (NPU). The at least one processor 120 may control input data to be processed based on the predefined operation rule stored in memory 110 or the artificial intelligence model. Alternatively, the at least one processor 120 may be a dedicated processor (or the processor dedicated to the artificial intelligence), and in this case, the at least one processor 120 may be designed to have a hardware structure specialized for processing a specific artificial intelligence model. For example, hardware specialized for processing the specific artificial intelligence model may be designed as a hardware chip, such as an application specific integrated circuit (ASIC) or a field programmable gate array (FPGA). In case that the at least one processor 120 is implemented as the dedicated processor, the at least one processor 120 may be implemented to include memory for implementing an embodiment of the disclosure, or may be implemented to include memory processing function for using external memory.

In another example, the memory 110 may store information on the artificial intelligence model including a plurality of layers. Here, storing the information on the artificial intelligence model may be storing various information related to an operation of the artificial intelligence model, for example, information on the plurality of layers included in the artificial intelligence model, a parameter used in each of the plurality of layers (for example, a filter coefficient or a bias).

It should be appreciated that the blocks in each flowchart and combinations of the flowcharts may be performed by one or more computer programs which include computer-executable instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g., a central processing unit (CPU)), a communication processor (CP, e.g., a modem), a graphical processing unit (GPU), a neural processing unit (NPU) (e.g., an artificial intelligence (AI) chip), a wireless-fidelity (Wi-Fi) chip, a Bluetooth™ chip, a global positioning system (GPS) chip, a near field communication (NFC) chip, connectivity chips, a sensor controller, a touch controller, a finger-print sensor controller, a display drive integrated circuit (IC), an audio CODEC chip, a universal serial bus (USB) controller, a camera controller, an image processing IC, a microprocessor unit (MPU), a system on chip (SoC), an IC, or the like.

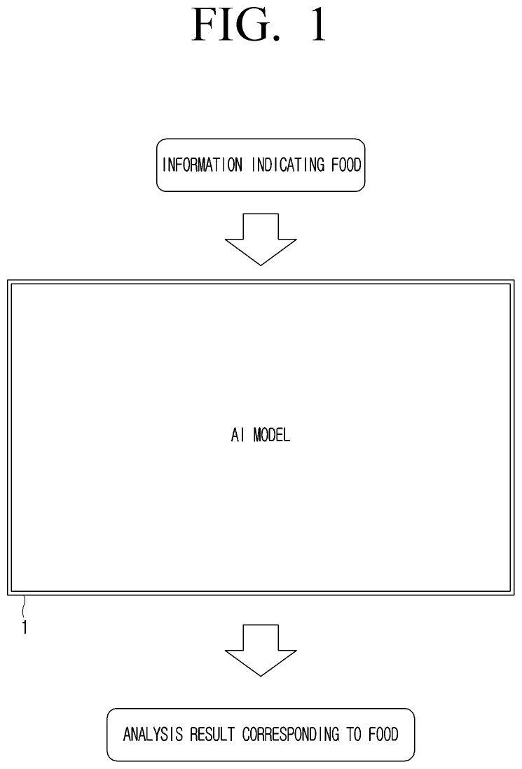

FIG. 1 is a view illustrating an artificial intelligence model 1 according to an embodiment of the disclosure.

Referring to FIG. 1, the electronic apparatus 100 may include the artificial intelligence model 1. The artificial intelligence model 1 may be described as a deep learning model, an artificial intelligence network, or the like. The artificial intelligence model 1 may receive information indicating a food as the input data. After receiving the information indicating the food as the input data, the artificial intelligence model 1 may provide an analysis result corresponding to the food.

A user input for the food that indicates the food may include at least one of text information indicating the food, image information indicating the food, or audio information indicating the food.

According to various embodiments of the disclosure, the electronic apparatus 100 may input food information itself into the artificial intelligence model 1.

According to various embodiments of the disclosure, the electronic apparatus 100 may perform a data preprocessing operation before inputting the food information into the artificial intelligence model 1. The electronic apparatus 100 may acquire a vector corresponding to the food information. The electronic apparatus 100 may acquire the vector corresponding to the food information by using a predetermined algorithm. The vector corresponding to the food information may be described as preprocessed data, food vector information, preprocessed data of the food information, preprocessed food information, or the like. The electronic apparatus 100 may input the vector corresponding to the food information into the artificial intelligence model 1.

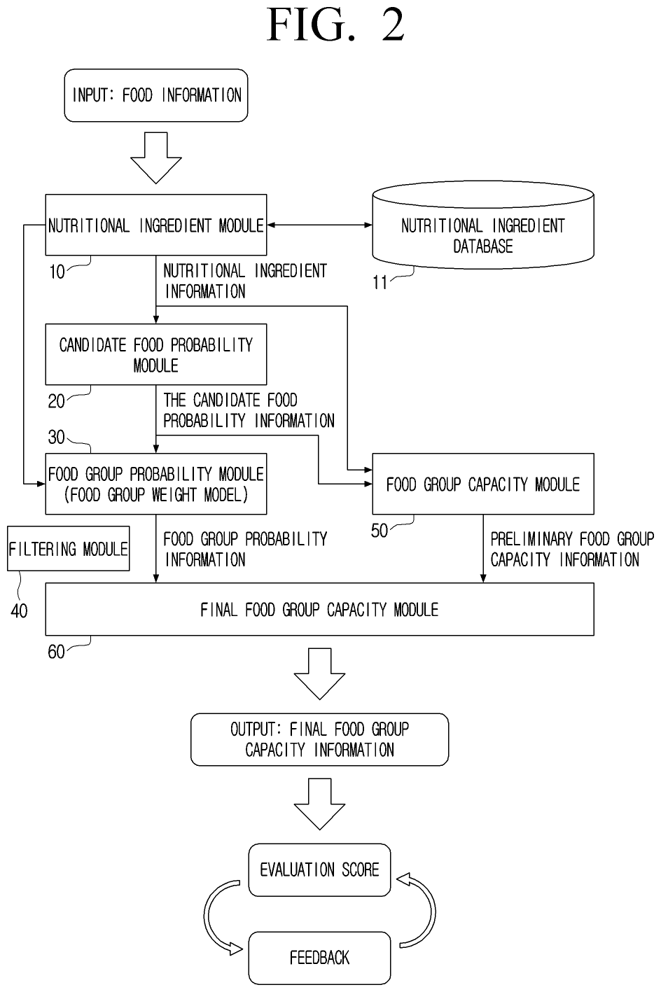

The artificial intelligence model 1 may include a plurality of modules. Its details are described with reference to FIG. 2. The artificial intelligence model 1 may generate various types of result information related to the food. The artificial intelligence model 1 may include a pre-trained model (or network). The electronic apparatus 100 may provide information related to an eating habit suitable for a user by using the artificial intelligence model 1.

FIG. 2 is a view illustrating at least one module included in an artificial intelligence model according to an embodiment of the disclosure.

Referring to FIG. 2, the electronic apparatus 100 may include at least one of a nutritional ingredient module 10, a candidate food probability module 20, a food group probability module 30, a filtering module 40, a food group capacity module 50, or a final food group capacity module 60. The electronic apparatus 100 may include a nutritional ingredient database 11.

According to an embodiment of the disclosure, each of the candidate food probability module 20, the food group probability module 30, and the food group capacity module 50 may be implemented as the artificial intelligence model. The other module may be implemented as a general calculation module.

The electronic apparatus 100 may acquire the user input for the food. The user input for the food may include various information indicating the food. The user input for the food may include at least one of the text information, the image information, or the audio information, indicating a specific food.

The electronic apparatus 100 may acquire the food information based the user input for the food. The food information may include the identification information and capacity information of the food. The identification information may indicate a food name. The capacity information may indicate the weight, volume, or the like of the food.

For example, the electronic apparatus 100 may acquire the identification information (or the name) indicating yogurt based on the user input.

For example, the electronic apparatus 100 may acquire 100 ml as a capacity of yogurt based on the user input.

A capacity may be expressed in a unit predetermined by the user. The user may use a unit “serving”, where “1 serving” may indicate 100 ml. The predetermined unit may differ depending on the food.

For example, the electronic apparatus 100 may receive the user input including one of the text information (e.g., 1 serving of yogurt), the image information (e.g., image data including yogurt), or the audio information (e.g., audio data including “1 serving of yogurt”).

According to various embodiments of the disclosure, a food information acquisition module may be used to acquire the food information. The electronic apparatus 100 may acquire the food information corresponding to the user input by inputting the user input for the food into the food information acquire module.

According to various embodiments of the disclosure, the user input may include the identification information and/or capacity information of the food. In case that the user input includes the identification information and/or capacity information of the food, the electronic apparatus 100 may directly acquire the food information based on the user input.

The electronic apparatus 100 may transmit the food information acquired based on the user input to the nutritional ingredient module 10.

The nutritional ingredient module 10 may receive the food information. The nutritional ingredient module 10 may acquire nutritional ingredient information based on the food information. The nutritional ingredient module 10 may acquire the nutritional ingredient information corresponding to the food information by using information included in the nutritional ingredient database 11.

The nutritional ingredient information may include at least one nutritional ingredient included in the food. The nutritional ingredient information may include a specific nutritional ingredient and a capacity corresponding to the specific nutritional ingredient. The nutritional ingredient module 10 may acquire the nutritional ingredient information based on the food information. The nutritional ingredient information may include a nutritional ingredient predetermined by the user. The nutritional ingredient may include a nutrient included in the food.

The nutritional ingredient may include an active ingredient having a nutritional effect in a human body. The nutritional ingredient information may be information related to the nutritional ingredient included in the food. The nutritional ingredient information may be a sub-concept of the food. The nutritional ingredient information may include a specific nutritional ingredient and a capacity value corresponding to the specific nutritional ingredient.

According to an embodiment of the disclosure, the nutritional ingredient may include at least one of energy (calories), protein, carbohydrates, sugar, dietary fiber, fat (or lipid), saturated fatty acid, monounsaturated fatty acid, polyunsaturated fatty acid, cholesterol, vitamins, minerals, or water. The vitamins may be classified by type, such as vitamins A, C, D, E, and K.

The capacity value may include a mass value. A unit of the mass value may be different based on the nutritional ingredient. The unit of the mass value may include at least one of mg, g, or kg.

The nutritional ingredient module 10 may receive the food information as the input data. The nutritional ingredient module 10 may acquire the nutritional ingredient information corresponding to the food information as its output data by using data stored in the nutritional ingredient database 11.

The nutritional ingredient module 10 may acquire the nutritional ingredient information by using a multi layer perceptron (MLP) of an artificial neural network. The MLP may include an input layer, a hidden layer, and an output layer. The hidden layer may include a plurality of layers. The food may not include a single ingredient, and the nutritional ingredient module 10 may thus perform a function of predicting various nutritional ingredients.

The input data of the nutritional ingredient module 10 may include at least one of image type data, audio type data, or text type data. The output data of the nutritional ingredient module 10 may include vector type data. The vector type data may include a vector indicating nutrient information of the food information. The output data may include a nutritional ingredient vector.

The nutritional ingredient module 10 may map the specific nutritional ingredient and the capacity value corresponding to the specific nutritional ingredient. The nutritional ingredient module 10 may acquire mapping information related to at least one nutritional ingredient as the output data. For example, the nutritional ingredient module 10 may acquire, as the output data, the mapping information (e.g., protein of 8.17 g) where the nutritional ingredient (e.g., protein) and the capacity value (e.g., 8.17 g) corresponding to the nutritional ingredient are mapped based on the input food information.

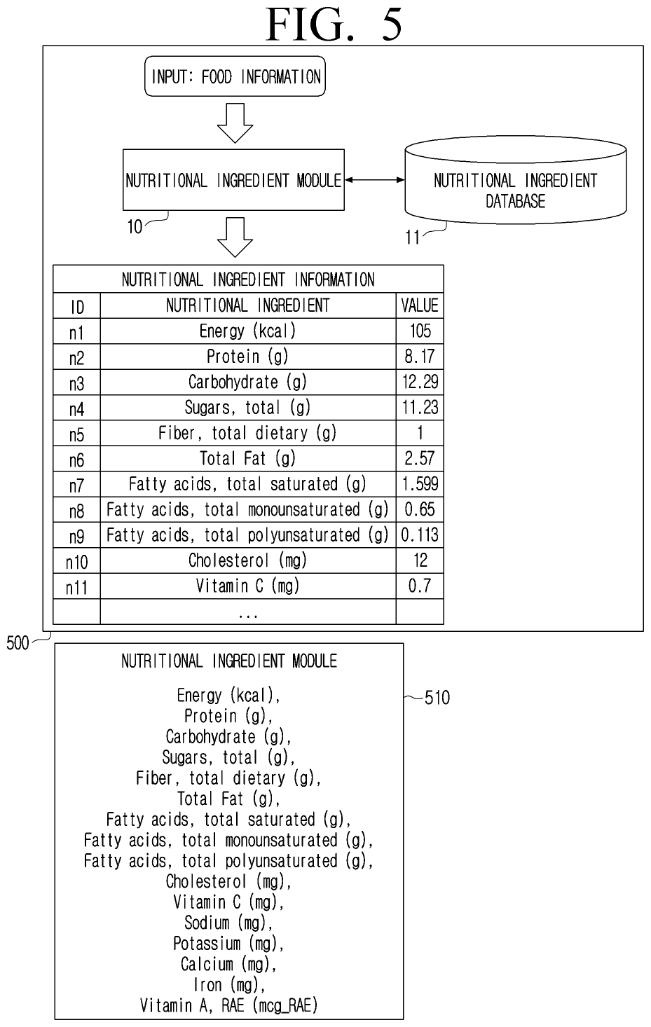

The nutritional ingredient information is described with reference to FIG. 5.

The nutritional ingredient module 10 may transmit the nutritional ingredient information to at least one of the candidate food probability module 20, the food group probability module 30, or the food group capacity module 50.

The candidate food probability module 20 may receive the nutritional ingredient information from the nutritional ingredient module 10. The candidate food probability module 20 may acquire candidate food probability information based on the nutritional ingredient information.

The candidate food may include at least one food for which it is difficult to accurately determine a specific food group by using the food information. The candidate food may include at least one food determined based on a user setting.

The candidate food may include a food for which classification accuracy of the food group is determined not to be high. The user may determine, as the candidate food, at least one food for which the classification accuracy or analysis accuracy is not high. The candidate food may be described as a reference food, a predetermined food, a user-set food, a filtered food, or the like.

The candidate food may include the reference food used to increase accuracy of food group probability information. It may be difficult to accurately determine the food group by using the food information that is highly likely to be determined as the candidate food. The candidate food probability module 20 may determine whether the food information is the candidate food to increase the accuracy of the food group probability information corresponding to the food information, and acquire the food group probability information by using a determination result.

The candidate food may indicate information used to acquire the food group probability information. The candidate food probability module 20 may acquire the food group probability information by further considering the candidate food probability information in addition to the nutritional ingredient information. The candidate food probability module 20 may acquire the food group probability information corresponding to the food information by further using the candidate food probability information.

Accuracy of acquiring the food group probability information by using both the nutritional ingredient information and the candidate food probability information may be higher than accuracy of acquiring the food group probability information by using only the nutritional ingredient information.

For example, the candidate food may include at least one of salad, dumplings, pasta, sandwich, or others. The candidate food may be changed based on the user setting.

The candidate food probability module 20 may calculate a probability value that the corresponding food information belongs to the candidate food by using only the nutritional ingredient information. The candidate food probability information may include mapping information where at least one candidate food and the probability value that the corresponding food information belongs to at least one candidate food are mapped.

The candidate food probability module 20 may receive the nutritional ingredient information as the input data. The candidate food probability module 20 may acquire the candidate food probability information as the output data.

The candidate food probability module 20 may acquire the candidate food probability information by using the multi layer perceptron (MLP) of the artificial neural network. The candidate food probability module 20 may include a prediction model implementing a classification model as the MLP. The MLP may include the input layer, the hidden layer, and the output layer. The hidden layer may include a plurality of layers. The hidden layer may include a layer using a leaky rectified linear unit (LReLU) function (or activation) and a layer using a Softmax function (or activation). The lowermost layer among the plurality of layers included in the hidden layer may be the layer using the LReLU function (or activation). The uppermost layer among the plurality of layers included in the hidden layer may be the layer using the Softmax function (or activation).

The input data used in the candidate food probability module 20 may include the vector type data indicating the nutrient information of the food information. The output data output from the candidate food probability module 20 may include a probability vector indicating a probability where the corresponding food information belongs to the candidate food.

The candidate food probability module 20 may map a specific candidate food and the probability value corresponding to the specific candidate food. The candidate food probability module 20 may acquire the mapping information related to at least one candidate food as the output data. For example, the candidate food probability module 20 may acquire, as the output data, the mapping information (e.g., salad of 0.02) where the candidate food (e.g., salad) and the probability value (e.g., 0.02) corresponding to the candidate food are mapped based on the input nutritional ingredient information.

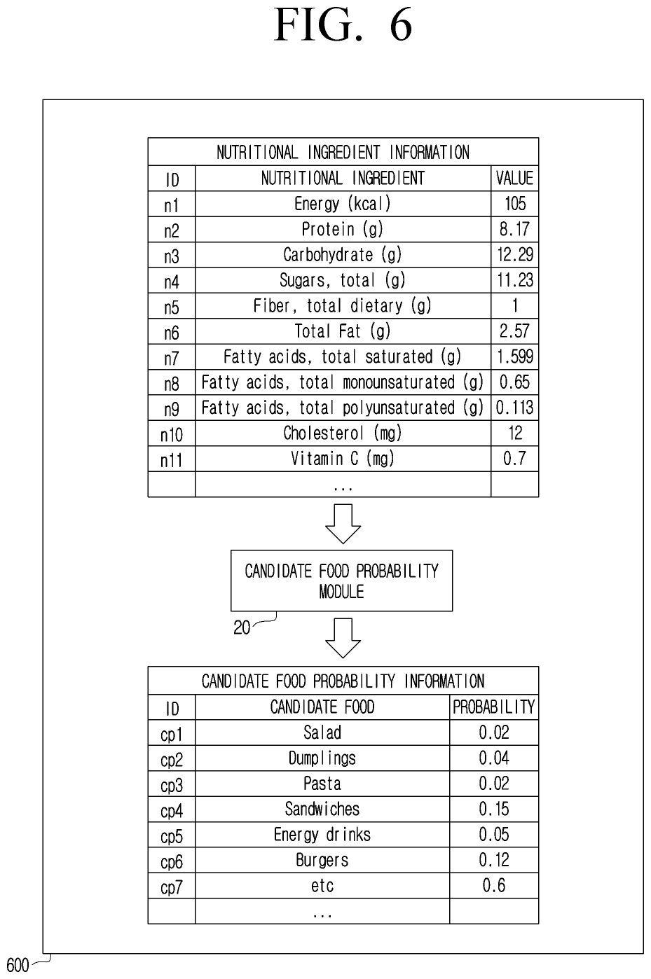

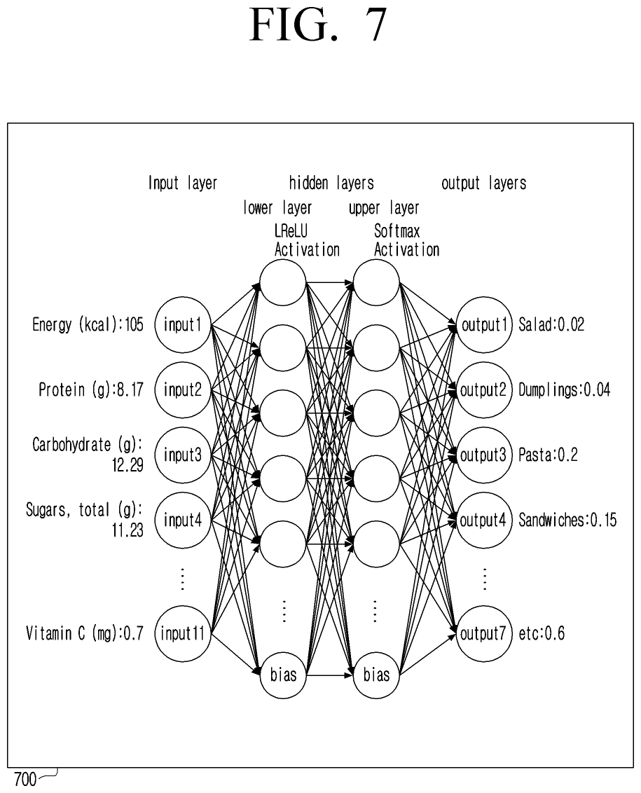

The candidate food probability information is described with reference to FIGS. 6 and 7.

The candidate food probability module 20 may transmit the candidate food probability information to at least one of the food group probability module 30 or the food group capacity module 50.

The food group probability module 30 may receive the nutritional ingredient information from the nutritional ingredient module 10. The food group probability module 30 may receive the candidate food probability information from the candidate food probability module 20. The food group probability module 30 may acquire the food group probability information based on at least one of the nutritional ingredient information or the candidate food probability information.

The food group may include a classification unit of the predetermined food. The food group may include at least one classification unit. The food group may include a unit indicating a specific category of the food. For example, yogurt (i.e., food) may be included in a dairy product (i.e., food group). The unit indicating the food group may be determined based on a predetermined reference in the user setting. The food group may be a concept different from the nutritional ingredient. The food group may be an upper concept of the food, and the nutritional ingredient may be a lower concept of the food. The food group may be the classification unit that may indicate a plurality of foods, and the nutritional ingredient may be an ingredient included in one food. For example, the food group may include at least one of the dairy products, beans-cereals, green vegetables, refined grains, plant-based proteins, seafood proteins, fruit juices, meat-poultry-egg proteins, other vegetables, starchy vegetables, fruits, or grains. The food group may be changed based on the user setting.

The food group may be described as food classification, a food classification unit, a foodstuff group, a food category, a food division unit, a predetermined group, or the like.

The food group probability information may include the probability value indicating a possibility that the food information is included in the specific food group. The food group probability module 30 may calculate the probability value that the food information belongs to the specific food group. The food group probability module 30 may acquire the food group probability information including the probability value indicating which food group the food information belongs to. The higher the probability value, the higher the possibility that the food information corresponds to the specific food group.

The food group probability module 30 may receive, as the input data, the nutritional ingredient information and the candidate food probability information. The food group probability module 30 may acquire the food group probability information as its output data.

The food group probability module 30 may acquire the food group probability information by using the multi layer perceptron (MLP) of the artificial neural network. The food group probability module 30 may include the prediction model implementing the classification model as the MLP. The MLP may include the input layer, the hidden layer, and the output layer. The hidden layer may include the plurality of layers. The hidden layer may include the layer using the LReLU function (or activation) and the layer using a Sigmoid function (or activation). The lowermost layer among the plurality of layers included in the hidden layer may be the layer using the LReLU function (or activation). The uppermost layer among the plurality of layers included in the hidden layer may be the layer using the Sigmoid function (or activation).

The nutritional ingredient information among the input data used by the food group probability module 30 may include the nutritional ingredient vector indicating the nutrient information of the food information. The candidate food probability information among the input data used by the food group probability module 30 may include the probability vector. The output data output from the food group probability module 30 may include the probability vector indicating the probability of the food group.

The food group probability module 30 may map the specific food group and the probability value corresponding to the specific food group. The food group probability module 30 may acquire the mapping information related to at least one food group as the output data. For example, the food group probability module 30 may acquire, as the output data, the mapping information (e.g., dairy product of 0.093) where the food group (e.g., dairy product) and the probability value (e.g., 0.93) corresponding to the food group are mapped based on the input nutritional ingredient information and candidate food probability information.

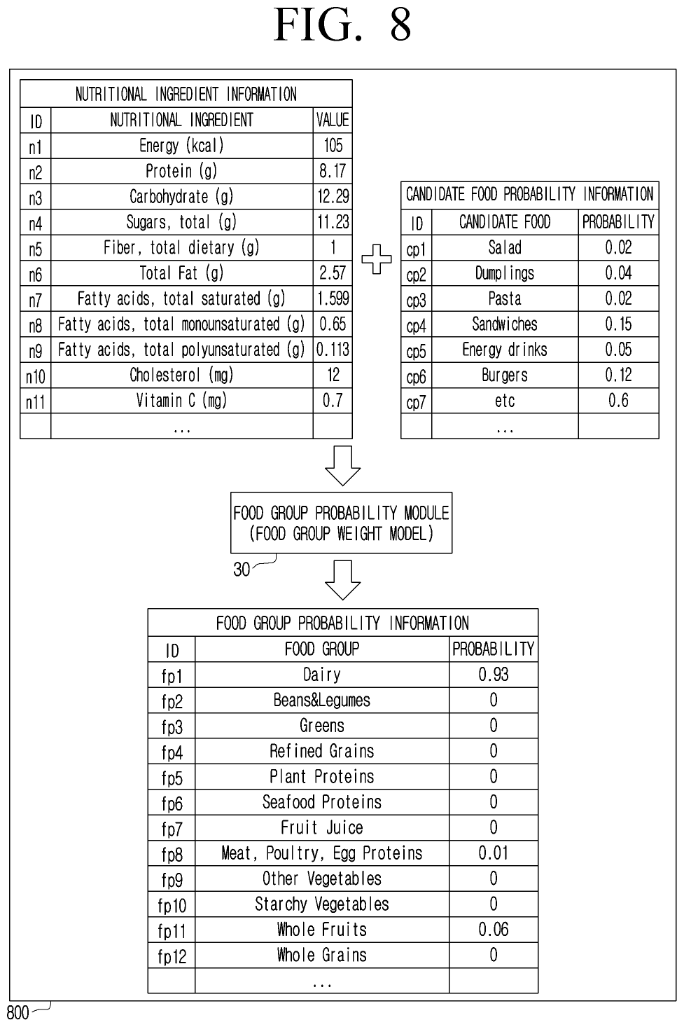

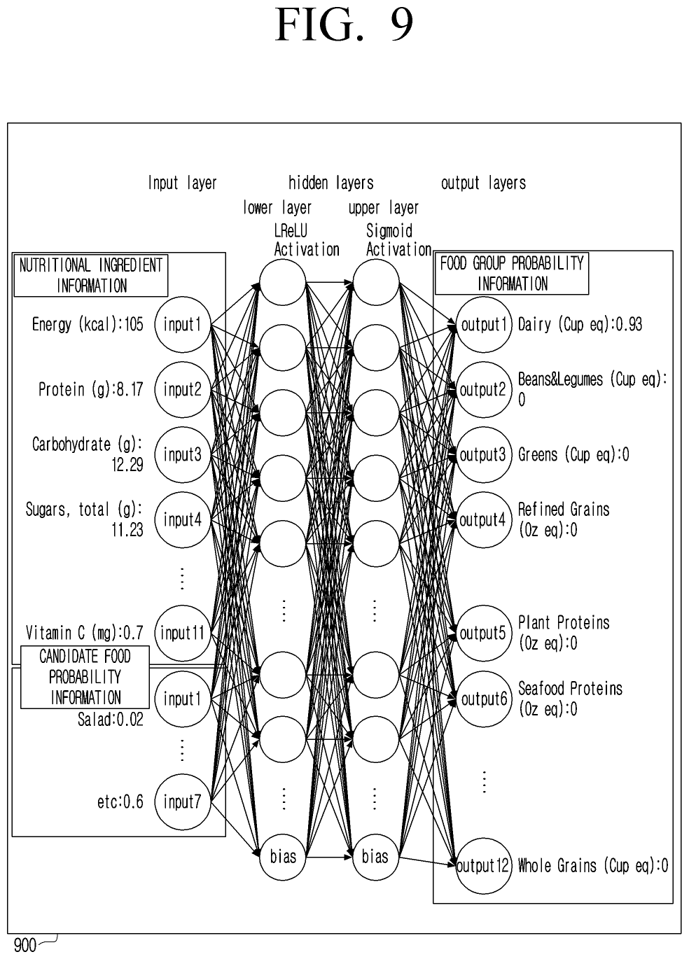

The food group probability information is described with reference to FIGS. 8 and 9.

The food group probability module 30 may transmit the food group probability information to the final food group capacity module 60.

The filtering module 40 may be a module filtering the food group probability information. The filtering module 40 may filter the food group probability information acquired from the food group probability module 30 based on a predetermined threshold value. The filtering module 40 may receive the food group probability information from the food group probability module 30. The filtering module 40 may remove (or filter) a corresponding probability value in case that the probability value included in the food group probability information is less than the threshold value.

The filtering module 40 may perform filtering for the food group probability information by removing the probability value less than the predetermined threshold value from the food group probability information, and acquire the filtered food group probability information as a filtering operation result.

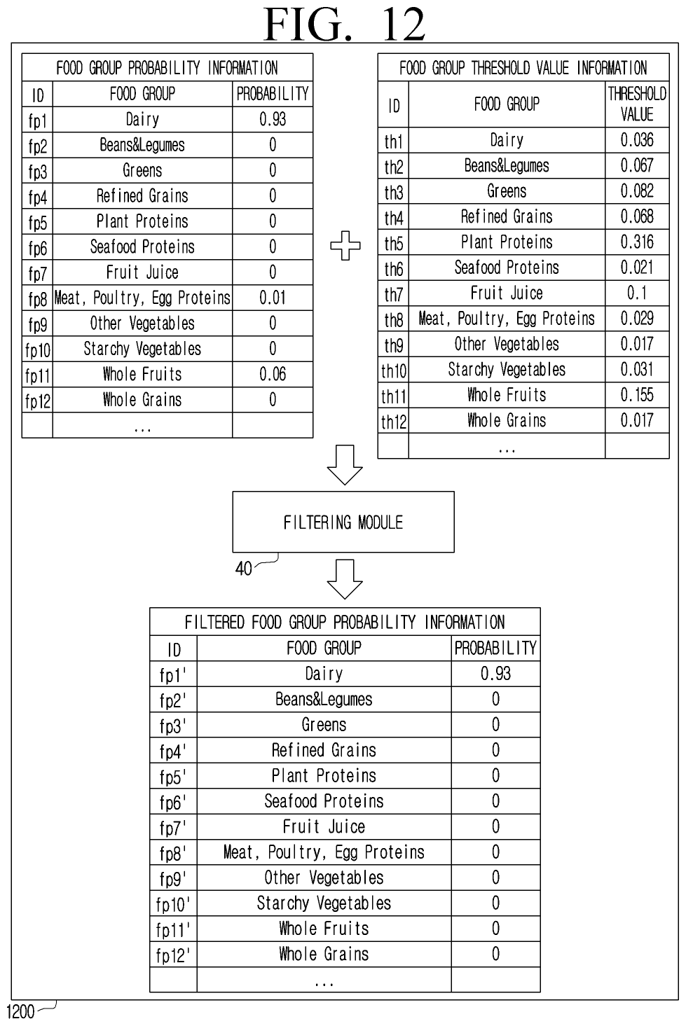

The determination result may have lower importance in case that the probability value is less than the predetermined threshold value. The filtering module 40 may remove (or filter) information on a food group whose probability value is less than the predetermined threshold value. The filtering module is described with reference to FIG. 12.

The filtering module 40 may transmit the filtered food group probability information to the final food group capacity module 60.

The food group capacity module 50 may receive the nutritional ingredient information from the nutritional ingredient module 10. The food group capacity module 50 may receive the candidate food probability information from the candidate food probability module 20. The food group capacity module 50 may acquire preliminary food group capacity information based on at least one of the nutritional ingredient information or the candidate food probability information.

The preliminary food group capacity information may be different from final food group capacity information. The preliminary food group capacity information may be described as first-type food group capacity information. The final food group capacity information may be described as second-type food group capacity information. The same food group may be defined in the preliminary food group capacity information and the final food group capacity information. Some meaningless information may be excluded from the final food group capacity information in that the filtering operation may be further reflected.

The preliminary food group capacity information may have low accuracy. The reason is that classification of the food information input by the user may not all be accurate. Therefore, the food group capacity module 50 may acquire the final food group capacity information by further considering the food group probability information in addition to the preliminary food group capacity information. The food group capacity module 50 may classify the information into preliminary information, final information, or the like to facilitate the classification in each operation.

The food group capacity module 50 may receive, as the input data, the nutritional ingredient information and the candidate food probability information. The food group capacity module 50 may acquire the preliminary food group capacity information as its output data.

The food group capacity module 50 may acquire the preliminary food group capacity information by using the multi layer perceptron (MLP) of the artificial neural network. The food group capacity module 50 may include a prediction model implementing a regression model as the MLP. The MLP may include the input layer, the hidden layer, and the output layer. The hidden layer may include the plurality of layers. The hidden layer may include the layer using the LReLU function (or activation) and the layer using the Softmax function (or activation). The lowermost layer among the plurality of layers included in the hidden layer may be the layer using the LReLU function (or activation). The uppermost layer among the plurality of layers included in the hidden layer may be the layer using the Softmax function (or activation).

The nutritional ingredient information among the input data used by the food group probability module 50 may include the nutritional ingredient vector indicating the nutrient information of the food information. The candidate food probability information among the input data used by the food group capacity module 50 may include the probability vector. The output data output from the food group capacity module 50 may include a capacity vector indicating a prediction capacity of the food group.

The food group probability module 50 may map the specific food group and a capacity value corresponding to the specific food group. The food group capacity module 50 may acquire the mapping information related to at least one food group as the output data. For example, the food group capacity module 50 may acquire, as the output data, the mapping information (e.g., dairy product of 0.33 cup) where the food group (e.g., dairy product) and the capacity value (e.g., 0.33 cup) corresponding to the food group are mapped based on the input nutritional ingredient information and candidate food probability information.

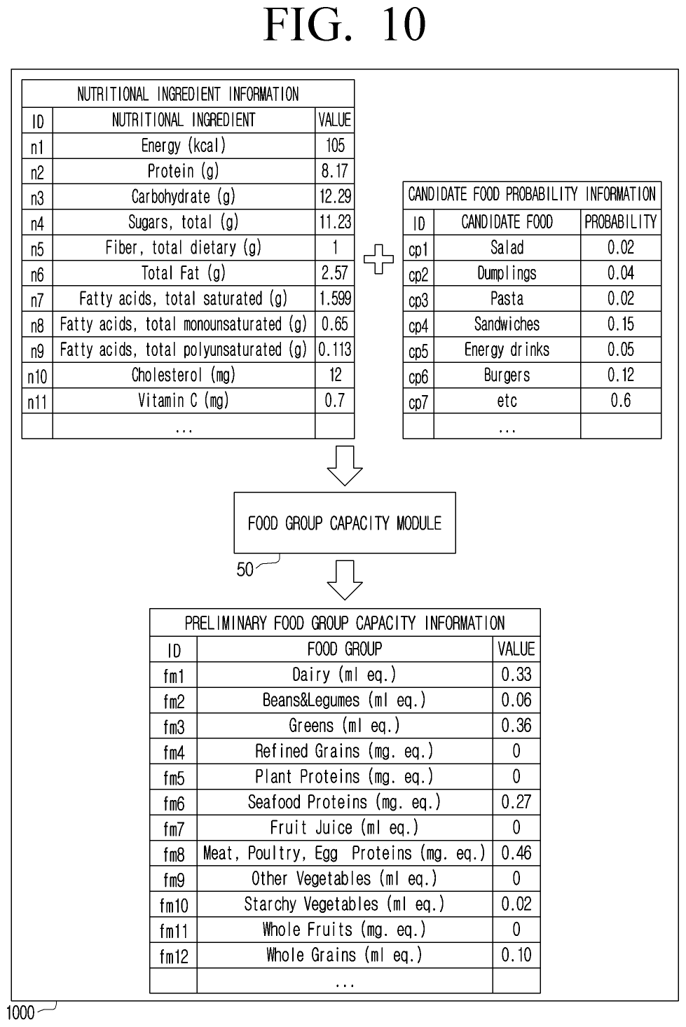

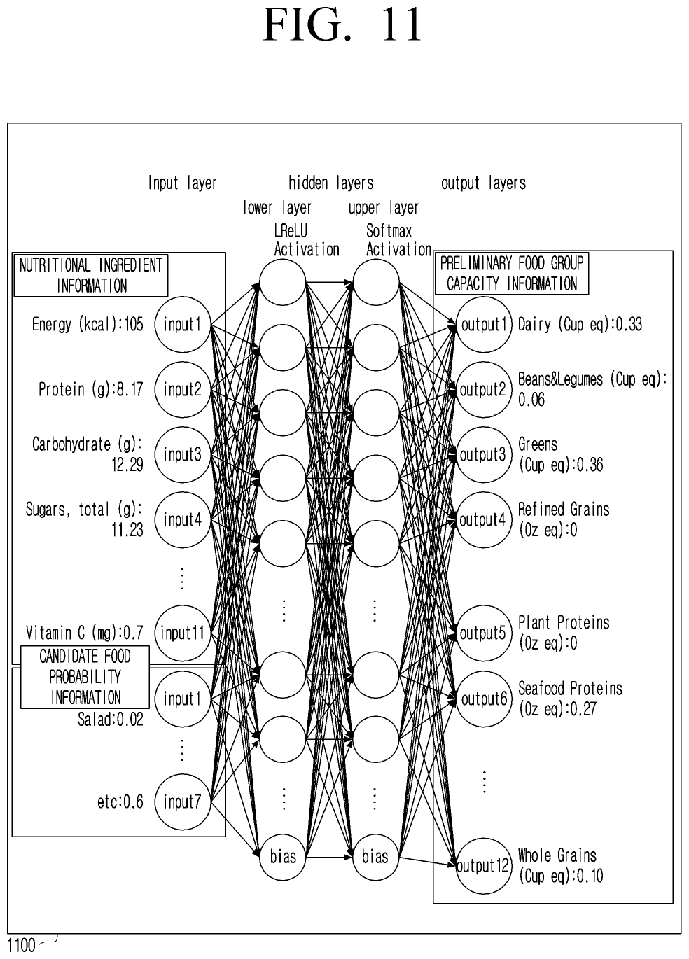

The preliminary food group capacity information is described with reference to FIGS. 10 and 11.

The electronic apparatus 100 may include a food group capacity database during its learning. In performing the learning operation, the electronic apparatus 100 and the food group capacity module 50 may use data on a capacity for each food group that is stored in the food group capacity database.

The food group capacity module 50 may transmit the preliminary food group capacity information to the final food group capacity module 60.

The final food group capacity module 60 may receive the food group probability information from the food group probability module 30. The final food group capacity module 60 may receive the filtered food group probability information from the filtering module 40. The final food group capacity module 60 may receive the preliminary food group capacity information from the food group capacity module 50. The final food group capacity module 60 may acquire the final food group capacity information based on at least one of the food group probability information or the preliminary food group capacity information. The final food group capacity information may be output data of the artificial intelligence model 1.

The capacity information may include a value indicating a capacity of a target. The capacity information may include at least one of mass information, weight information, or volume information. The final food group capacity information may include the specific food group and a capacity corresponding to the specific food group. The specific food group and the capacity corresponding to the specific food group may be mapped and then included in the final food group capacity information.

The final food group capacity module 60 may couple the food group probability information with the preliminary food group capacity information, thereby acquiring a capacity value corresponding to each of final food groups.

The final food group capacity module 60 may select the final food group from the plurality of food groups. The final food group capacity module 60 may determine the final food group by comparing the food group probability information with the threshold value. The final food group capacity module 60 may determine (or select), as the final food group, the food group corresponding to a probability value of the threshold value or more among the plurality of food groups. The final food group capacity module 60 may acquire the final food group capacity information based on the capacity information corresponding to the final food group among the preliminary food group capacity information.

The final food group capacity module 60 may receive, as the input data, the food group probability information and the preliminary food group capacity information. The final food group capacity module 60 may acquire the final food group capacity information as its output data.

The final food group capacity module 60 may use a “threshold-moving” method of acquiring final capacity information by using the threshold value. The final food group capacity module 60 may exclude, from the coupling operation, the food group with a probability value less than the threshold value among the food group probability information. The final food group capacity module 60 may acquire the final food group capacity information by considering only the food group with the probability value of the threshold value or more.

The “threshold-moving” method may include an operation of converting a class label based on a specific threshold value. For example, the final food group capacity module 60 may classify, as class “zero” in case that a target value is less than the threshold value. The final food group capacity module 60 may classify, as class “1” in case that the target value is the threshold value or more. The final food group capacity module 60 may acquire an optimal threshold value by using an “F-measure” method. The “F-measure” method may include an operation of calculating harmonic means of precision and recall.

According to an embodiment of the disclosure, the threshold value may be a predetermined value. For example, the threshold value may be 0.5.

According to an embodiment of the disclosure, the threshold value may be different for each food group. For example, a threshold value of the dairy product may be 0.036 and a threshold value of the beans-cereals may be 0.067. The final food group capacity module 60 may acquire the output data by using the capacity information (e.g., 0.33) of the dairy product in case that the probability value (e.g., 0.93) of the dairy product is the threshold value (e.g., 0.036) or more. The final food group capacity module 60 may acquire the output data without using the capacity information (e.g., 0.06) of the beans-cereals in case that the probability value of the beans-cereals is “zero” and is less than the threshold value (e.g., 0.36).

The food group probability information among the input data used by the final food group capacity module 60 may include the probability vector. The preliminary food group capacity information among the input data used by the final food group capacity module 60 may include the capacity vector. The output data output from the final food group capacity module 60 may include the capacity vector indicating the capacity value corresponding to each of the final food group.

The final food group capacity module 60 may map the final food group and the capacity value corresponding to the final food group. The final food group capacity module 60 may acquire the mapping information related to at least one final food group as the output data. For example, the food group capacity module 60 may acquire, as the output data, the mapping information (e.g., dairy product of 0.33 cup) where the final food group (e.g., dairy product) and the capacity value (e.g., 0.33 cup) corresponding to the food group are mapped based on the food group probability information and the preliminary food group capacity information.

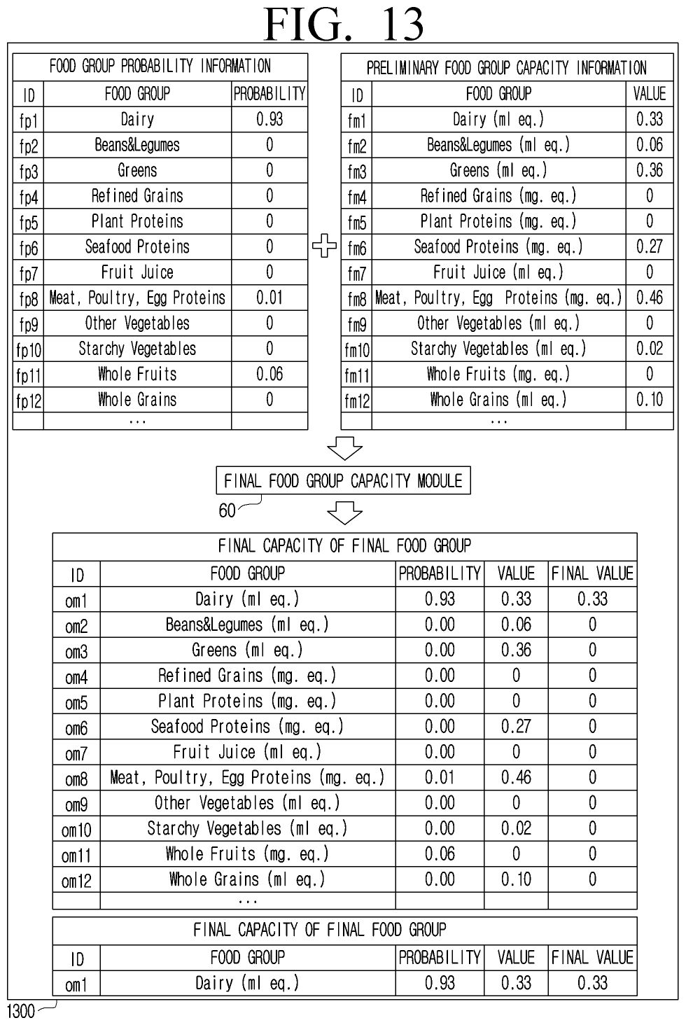

The final food group capacity module 60 is described with reference to FIG. 13.

The electronic apparatus 100 may calculate an evaluation score based on the final food group capacity information. The electronic apparatus 100 may update (or change) the evaluation score based on user feedback corresponding to the evaluation score.

Some operations performed or some modules in the description above may be changed based on an embodiment.

For example, each of the nutritional ingredient module 10, the candidate food probability module 20, the food group probability module 30, the filtering module 40, the food group capacity module 50, and the final food group capacity module 60 may be implemented as the artificial intelligence model. Each module may be implemented as the artificial intelligence model.

For example, at least one of the nutritional ingredient module 10, the candidate food probability module 20, the food group probability module 30, the filtering module 40, the food group capacity module 50, the final food group capacity module 60, or the nutritional ingredient database 11 may be included in the artificial intelligence model 1.

For example, the electronic apparatus 100 may transmit the food group probability information to the final food group capacity module 60 without performing the filtering function by the filtering module 40.

For example, the filtering module 40 may be included in the food group probability module 30.

For example, the preliminary food group capacity information may be described as first capacity information, first-type capacity information, and sub-capacity information. The final food group capacity information may be described as second capacity information, second-type capacity information, and main capacity information.

For example, the electronic apparatus 100 may store a first data set including a structure of the nutritional ingredient occupied by a specific ingredient per unit capacity (e.g., 100 g) of the specific ingredient. The electronic apparatus 100 may store a second data set indicating a capacity of each ingredient necessary to make the specific food. The electronic apparatus 100 may acquire additional data by using the first data set and the second data set. The electronic apparatus 100 may use the additional data for the learning operations of the food group probability module 30 and the food group capacity module 50.

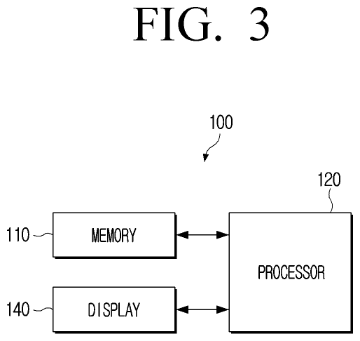

FIG. 3 is a block diagram illustrating an electronic apparatus according to an embodiment of the disclosure.

Referring to FIG. 3, the electronic apparatus 100 may include at least one of the memory 110, the at least one processor 120, or a display 140.

The electronic apparatus 100 may include various devices including the display. The electronic apparatus 100 may be an electronic whiteboard, a television (TV), a desktop personal computer personal computer (PC), a laptop PC, a smartphone, a tablet PC, a server, or the like. The above-described example is only an example to describe the electronic apparatus, and is not necessarily limited to the above-described device.

The at least one processor 120 may perform overall control operations of the electronic apparatus 100. The at least one processor 120 may function to control the overall operations of the electronic apparatus 100.

The at least one processor 120 may receive the user input for the food, acquire the food information corresponding to the food from the received user input, acquire the nutritional ingredient information of the acquired food information by inputting the acquired food information into the nutritional ingredient module, acquire the candidate food probability information by inputting the nutritional ingredient information into the candidate food probability module, acquire the food group probability information and the preliminary food group capacity information by inputting the acquired nutritional ingredient information and the acquired candidate food probability information respectively into the food group probability module and the food group capacity module, acquire the filtered food group probability information by removing the probability value less than the threshold value from the acquired food group probability information, and acquire the final food group capacity information by coupling (or by combining) the filtered food group probability information with the preliminary food group capacity information.

The user input for the food may be described as the user input for selecting the food, the user input indicating the food, the user input about the food, the user input related to the food, or the like.

The user input for the food may include at least one of the text information, the image information, or the audio information, indicating the specific food.

According to various embodiments of the disclosure, the user input may include the text information, the image information, or the audio information. The at least one processor 120 may acquire the food information (or detailed food information) on the food based on the user input. The at least one processor 120 may receive the user input including one of the text information (e.g., 1 serving of yogurt), the image information (e.g., image data including yogurt), or the audio information (e.g., audio data including “1 serving of yogurt”). The at least one processor 120 may acquire the food information based on the received user input. The food information may include the identification information of the food (e.g., yogurt) or the capacity information of the food (e.g., 100 ml).

According to various embodiments of the disclosure, the food information acquisition module may be used to acquire the food information. The at least one processor 120 may input the user input for the food to the food information acquisition module to acquire the food information corresponding to the user input.

The at least one processor 120 may acquire (or receive) the user input related to the food. The user input related to the food may include at least one of a name related to the specific food or a capacity of the specific food.

The nutritional ingredient information may include at least one nutritional ingredient included in the food. The nutritional ingredient information may include the specific nutritional ingredient and the capacity corresponding to the specific nutritional ingredient. The at least one processor 120 may acquire the nutritional ingredient information based on the food information. The nutritional ingredient information may include the nutritional ingredient predetermined by the user. The nutritional ingredient may include the nutrient included in the food.

The at least one processor 120 may acquire the candidate food probability information based on the nutritional ingredient information. The candidate food may include at least one predetermined food among the plurality of foods.

The at least one processor 120 may determine whether the food information is the candidate food to increase the accuracy of the food group probability information corresponding to the food information, and acquire the food group probability information by using the determination result.

The at least one processor 120 may acquire the food group probability information by further considering the candidate food probability information in addition to the nutritional ingredient information. The at least one processor 120 may acquire the food group probability information corresponding to the food information by further using the candidate food probability information.

The at least one processor 120 may calculate the probability value that the corresponding food information belongs to the candidate food by using only the nutritional ingredient information. The candidate food probability information may include the mapping information where at least one candidate food and the probability value that the corresponding food information belongs to at least one candidate food are mapped.

The at least one processor 120 may acquire the final food group capacity information based on at least one of the nutritional ingredient information or the candidate food probability information. The capacity information may include the specific food group and the capacity corresponding to the specific food group. The final food group capacity information may include the capacity corresponding to the specific food group. The final food group capacity information may include the mapping information where the specific food group and the capacity corresponding to the specific food group are mapped.

The at least one processor 120 may acquire food group threshold value information including the threshold value corresponding to each of the plurality of food groups, and acquire the filtered food group probability information by removing the probability value less than the threshold value included in the threshold value information from the food group probability information acquired for each of the plurality of food groups.

The at least one processor 120 may calculate the probability value that the food information belongs to the specific food group. The at least one processor 120 may acquire the food group probability information including the probability value indicating which the food group the food information belongs to.

The at least one processor 120 may perform the filtering by removing the probability value less than the threshold value from the food group probability information, acquire the filtered food group probability information as a filtering operation result, and acquire the final food group capacity information based on the filtered food group probability information.

The at least one processor 120 may remove (or filter) the information on the food group whose probability value is less than the threshold value.

The at least one processor 120 may acquire the final food group capacity information by using the filtered result information.

The at least one processor 120 may perform the filtering operation based on the food group threshold value information including the threshold value corresponding to each of the plurality of food groups. A different threshold value may correspond to each food group. Its details are described with reference to FIG. 12.

The at least one processor 120 may acquire the preliminary food group capacity information indicating capacity information for each of the plurality of food groups by inputting the nutritional ingredient information and the candidate food probability information into the food group capacity module.

The at least one processor 120 may acquire the final food group capacity information by further considering the food group probability information in addition to the preliminary food group capacity information. The at least one processor 120 may classify the information into the preliminary information, the final information, or the like to facilitate the classification in each operation. The preliminary food group capacity information may be described as the first food group capacity information (or the first-type food group capacity information), and the final food group capacity information may be described as the second food group capacity information (or the second-type food group capacity information).

The preliminary food group capacity information is described with reference to FIGS. 10 and 11.

The at least one processor 120 may update the user history indicating the user eating habit based on the final food group capacity information.

The at least one processor 120 may store, in the memory, the user history including accumulated capacity information for each of the plurality of food groups consumed by the user during a predetermined time period, and update the user history by accumulating the capacity value for each of the plurality of food groups included in the final food group capacity information.

The at least one processor 120 may acquire score information related to the user eating habit by comparing reference capacity information with the accumulated capacity information for each of the plurality of food groups included in the updated user history.

The user history may include various information related to the food consumed by the user.

The at least one processor 120 may accumulate the food information and the final food group capacity information corresponding to the food information for the predetermined time period. For example, the at least one processor 120 may accumulate a capacity of the dairy product consumed by the user during a week. The predetermined time period may be changed based on the user setting. The predetermined time period may include at least one of a daily basis, a weekly basis, a monthly basis, or a yearly basis.

The user history may include at least one of information on time in case that the user consumes the food, the food name, or a food capacity. The information stored in the user history may be accumulated data. The accumulated data may be described as statistical data.

The user history may include information corresponding to a specific user. For example, only information related to a first user may be stored in the user history. The user history may also be different for a different user.

In case of receiving the food information, the at least one processor 120 may acquire the final food group capacity information corresponding to the food information. The at least one processor 120 may update a pre-stored user history based on the final food group capacity information. The at least one processor 120 may change the user history to reflect new food information.

The at least one processor 120 may acquire the score information related to the user eating habit based on the updated user history.

The at least one processor 120 may calculate the score information indicating a comprehensive result related to the eating habit to the user. The at least one processor 120 may extract the information indicating the user eating habit from the user history. The at least one processor 120 may calculate the score information indicating a degree to which the user eating habit is correct based on the extracted information. The at least one processor 120 may provide the score information.

The score information may include at least one of a user eating habit score, a user age, or an average score of the user age.

The user eating habit score may indicate a score related to the eating habit calculated based on the information included in the user input. The eating habit score may be calculated by comprehensively considering the foods the user consumes. The eating habit score may indicate a score corresponding to the predetermined time period (e.g., one day, one week, one month, or one year).

The reference capacity information may include a capacity for each of the food groups that the user is required to consume. For example, the reference capacity information may include an essential food group that the user is required to consume on the daily basis and the capacity value corresponding to the essential food group. The reference capacity information may include a required capacity value corresponding to each of the plurality of food groups.

The user age may be described as user age information. The user age may include a user current age.

The average score of the user age may be provided by an external device (or external server) collecting data from the plurality of users. The average score of the user age may include an average eating habit score of people of the same age as the user age.

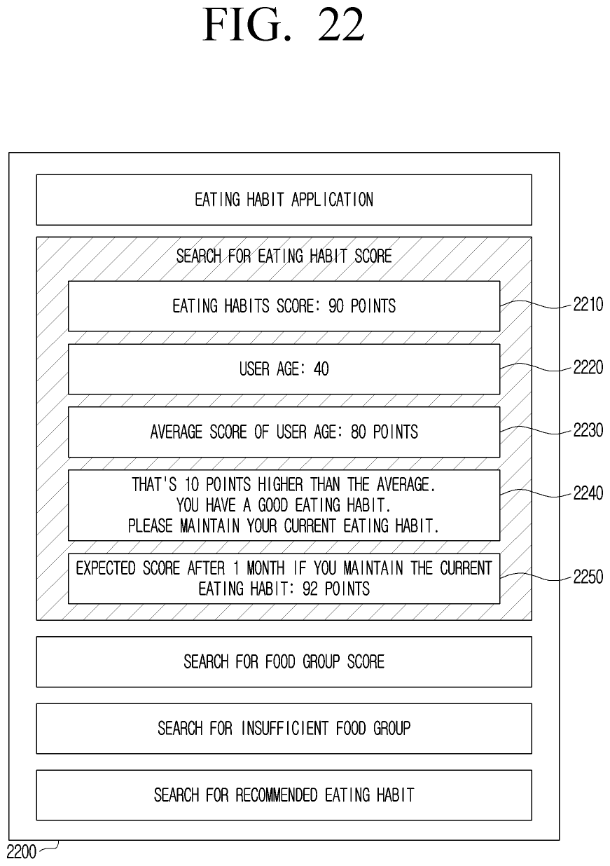

The score information is described with reference to FIG. 22.

The at least one processor 120 may provide the guide information indicating a recommended eating habit based on the updated user history, and the guide information may include at least one of information related to an insufficient food group or information related to an excessively consumed food group.

The at least one processor 120 may provide the guide information indicating the recommended eating habit for a first food group by comparing a first capacity value of the first food group that is included in the updated user history with a first reference value corresponding to the first food group that is included in the reference capacity information.

The user history may include the capacity value for each of the plurality of food groups. The capacity value for each of the food group may include at least one of an accumulated capacity value or an average capacity value, acquired during the predetermined time period. The average capacity value may be a value calculated based on a daily average, a weekly average, a monthly average, or the like.

The at least one processor 120 may compare a capacity value of a specific first food group among the plurality of food groups with the first reference value corresponding to the first food group. The at least one processor 120 may acquire the capacity value of the first food group based on the user history. The at least one processor 120 may acquire the first reference value corresponding to the first food group based on the reference capacity information. The at least one processor 120 may acquire the score information by comparing the first capacity value with the first reference value.

The at least one processor 120 may determine that the user consumes the minimum required capacity in case that the first capacity value is the first reference value or more.

The at least one processor 120 may determine that the user does not consume the minimum required capacity in case that the first capacity value is less than the first reference value.

The at least one processor 120 may acquire a difference value between the first capacity value and the first reference value. The less the difference value, the more the calculated eating habit score (or score information).

The at least one processor 120 may determine the recommended eating habit for the first food group based on the calculated score information.

The electronic apparatus 100 may further include the display, and the at least one processor 120 may control the display to display the guide screen including the food information and the final food group capacity information.

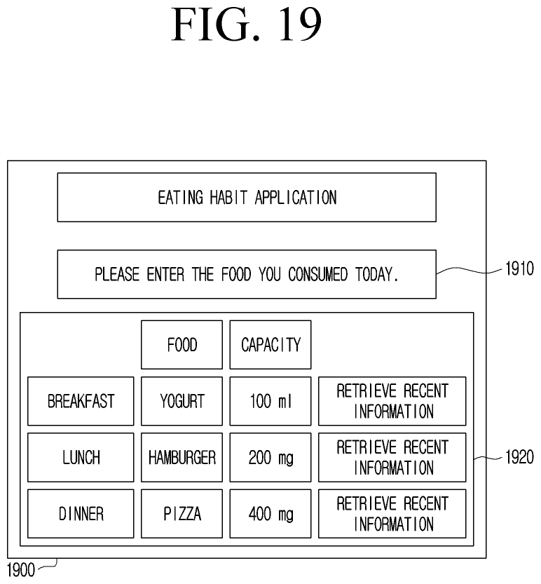

The guide screen or the guide information are described with reference to FIGS. 19 to 27.

According to various embodiments of the disclosure, in case of receiving the food information, the at least one processor 120 may determine whether the final food group capacity information corresponding to the food information exists in the database. The at least one processor 120 may acquire the final food group capacity information by calculating the nutritional ingredient information, the candidate food probability information, the food group probability information, the preliminary food group capacity information, or the like in case that the final food group capacity information corresponding to the food information does not exist in the database.

The electronic apparatus 100 may use the nutritional ingredient database to predict the food group of the food based on a user dietary history or the nutritional ingredient information, and calculate a user dietary history score by using predicted food group information.

There may be a case where dietary quality is unable to be measured because there is insufficient data to classify the food group. The electronic apparatus 100 may use machine learning to convert foods and drinks into the food group and score the user dietary history.

The electronic apparatus 100 may quantify whether the user consumes a healthy diet only by a diet input. The electronic apparatus 100 may guide the user to a healthy lifestyle habit by using a scored activity indicator, and provide feedback to further increase the score.

The electronic apparatus 100 may provide an algorithm for converting the user dietary history and the nutrient information to a food group format.

The electronic apparatus 100 may enable the user to acquire the food group information of the food where 10 types of food group information are not in the database, and make dietary evaluation possible based thereon.

The electronic apparatus 100 may generate the final food group capacity information by using the 10 types of food group information (e.g., total fruits, whole fruits, total vegetables, vegetables and beans, whole grains, total protein foods, seafood and vegetable proteins, dairy products, refined grains, or added sugars). The electronic apparatus 100 may generate the final food group capacity information based on an indicator reflecting a dietary guideline or a food recommendation.

According to various embodiments of the disclosure, the electronic apparatus 100 may identify a user dietary pattern through analysis of the derived statistical data, and utilize the dietary pattern for dietary coaching.

In providing a dietary pattern analysis, the electronic apparatus 100 may provide score change trend information for each predetermined time period (for each day/month/6 months/1 year).

The electronic apparatus 100 may provide statistical information on an interrelationship by providing user profile information (e.g., weight information).

In a dietary coaching method, the electronic apparatus 100 may provide the score for a degree of meeting a balance for each specific food group/nutrient. The electronic apparatus 100 may recommend foods that may improve the balance.

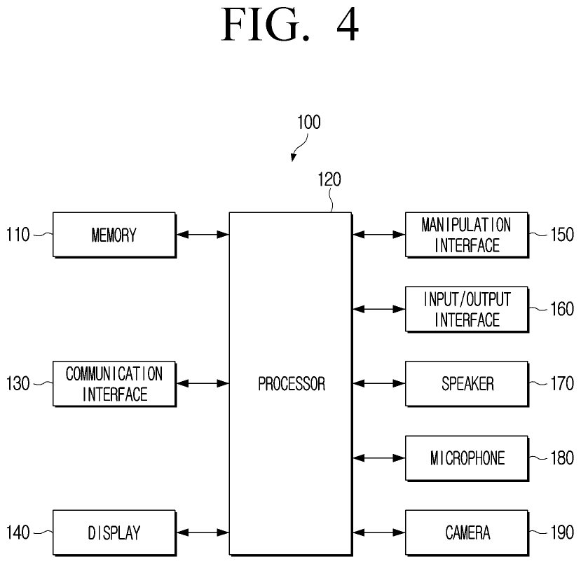

FIG. 4 is a block diagram illustrating a specific configuration of an electronic apparatus of FIG. 3 according to an embodiment of the disclosure.

Referring to FIG. 4, the electronic apparatus 100 may include at least one of the memory 110, the at least one processor 120, a communication interface 130, the display 140, a manipulation interface 150, and an input/output interface 160, a speaker 170, a microphone 180, or a camera 190.

The memory 110 may be implemented as internal memory, such as read-only memory (ROM) (e.g., electrically erasable programmable read-only memory (EEPROM) or random access memory (RAM), included in the at least one processor 120, or as memory separate from the at least one processor 120. In this case, the memory 110 may be implemented in the form of memory embedded in the electronic apparatus 100 or in the form of memory detachable from the electronic apparatus 100, based on a data storage purpose. For example, data for driving the electronic apparatus 100 may be stored in the memory embedded in the electronic apparatus 100, and data for the extended function of the electronic apparatus 100 may be stored in the memory detachable from the electronic apparatus 100.

The memory 110 may store at least one instruction. The at least one processor 120 may perform various operations based on the instruction stored in the memory 110.

The at least one processor 120 may be implemented as a digital signal processor (DSP) that processes a digital signal, a microprocessor, or a time controller (TCON). However, the at least one processor 120 is not limited thereto, and may include one or more of a central processing unit (CPU), a micro controller unit (MCU), a micro processing unit (MPU), a controller, an application processor (AP), a graphics-processing unit (GPU), a communication processor (CP), or an advanced reduced instruction set computer (RISC) machine (ARM) processor, or may be defined by these terms. The at least one processor 120 may be implemented as a system-on-chip (SoC) or a large scale integration (LSI), in which a processing algorithm is embedded, or may be implemented in the form of a field programmable gate array (FPGA). The at least one processor 120 may perform various functions by executing computer executable instructions stored in the memory.

The communication interface 130 is a component communicating with various types of external devices by using various types of communication methods. The communication interface 130 may include a wireless communication module or a wired communication module. Each communication module may be implemented in the form of at least one hardware chip.

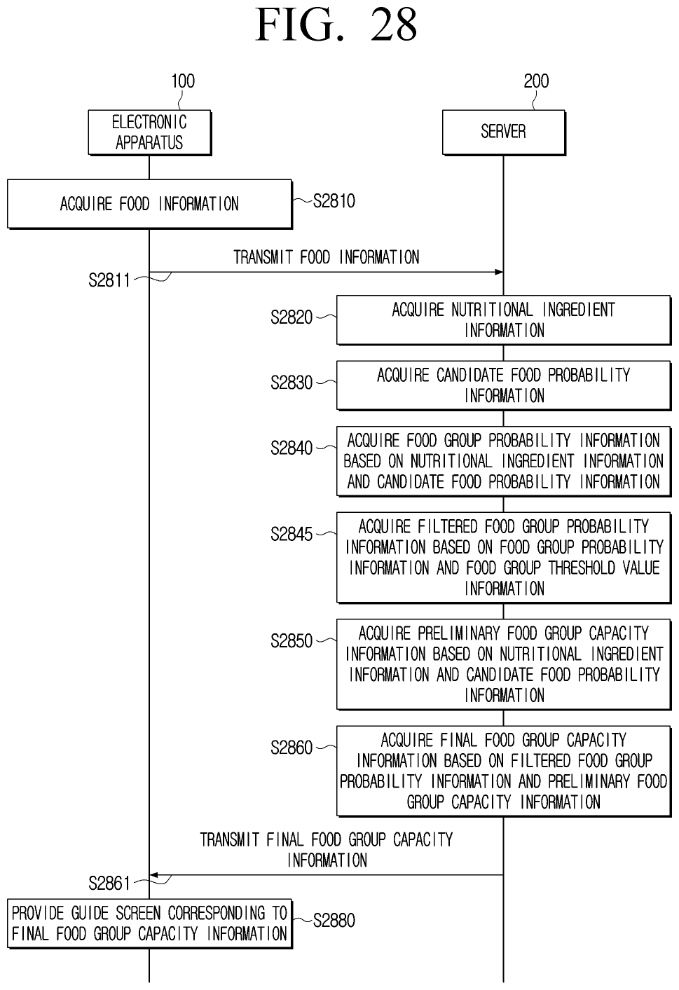

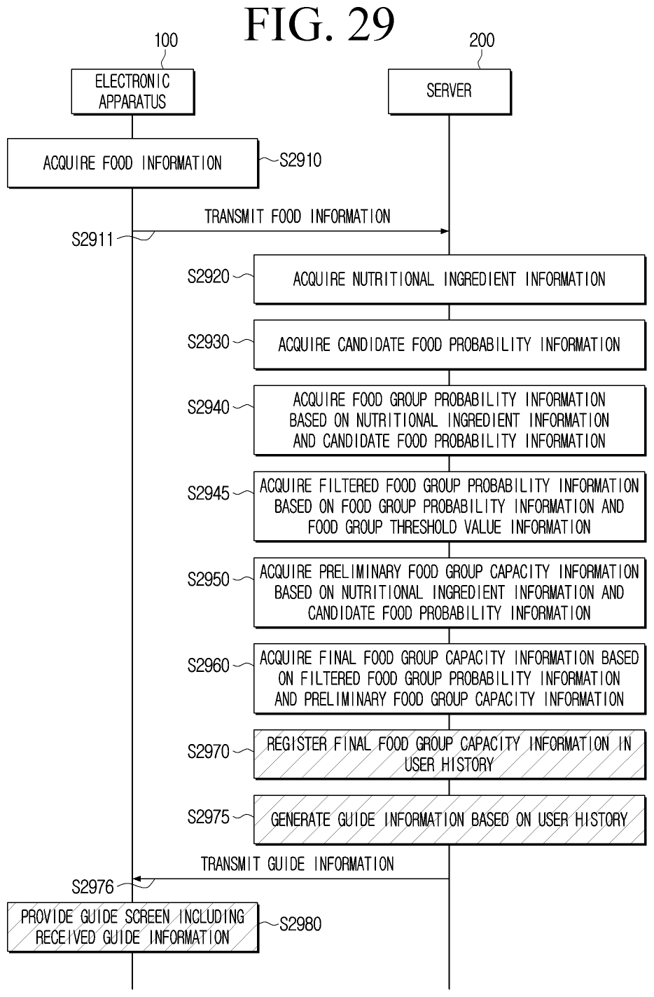

The electronic apparatus 100 may transmit the food information to a server 200 through the communication interface 130. The electronic apparatus 100 may receive the final food group capacity information from the server 200 through the communication interface 130. The electronic apparatus 100 may receive the guide information from the server 200 through the communication interface 130. Its details are described with reference to FIGS. 28 and 29.

The display 140 may be implemented as various types of displays, such as a liquid crystal display (LCD), an organic light emitting diode (OLED) display, a plasma display panel (PDP).

The electronic apparatus 100 may provide the guide screen including an analysis process related to the food information and the analysis result through the display 140. Its details are described with reference to FIGS. 19 to 29.

The manipulation interface 150 may be implemented as a device, such as a button, a touch pad, a mouse, or a keyboard, or may be implemented as a touch screen capable of also performing a manipulation input function in addition to the above-described display function. The button may be any of various types of buttons, such as a mechanical button, a touch pad, or a wheel, which is disposed in any region of a body appearance of the electronic apparatus 100, such as its front surface, side surface, or rear surface.

The electronic apparatus 100 may acquire the user input through the manipulation interface 150. The electronic apparatus 100 may acquire the user input for searching for specific information or executing a specific command through the manipulation interface 150.

The electronic apparatus 100 may acquire the user input indicating the food information through the manipulation interface 150. The electronic apparatus 100 may acquire the user input including the text information indicating the food information or the user command to input the image information (e.g., an image file) including the food information through the manipulation interface 150.

The input/output interface 160 may be any of a high definition multimedia interface (HDMI), a mobile high-definition link (MHL), a universal serial bus (USB), a display port (DP), a thunderbolt, a video graphics array (VGA) port, a red-green-blue (RGB) port, a D-subminiature (D-SUB), or a digital visual interface (DVI).

The speaker 170 may be a component outputting not only various audio data but also various notification sounds or voice messages.

The microphone 180 is a component receiving a user voice or other sounds to convert the same into the audio data.

The camera 190 may be a component capturing a subject and generating a captured image. Here, the captured image may include both moving images and still images. The camera 190 may acquire an image for at least one external device, and may be implemented as a camera, a lens, an infrared sensor, or the like.

The electronic apparatus 100 may acquire the image information indicating the food information through the camera 190. The electronic apparatus 100 may acquire images capturing the food as the target by the camera 190. The captured image may include the food. The captured image may be used as the food information, and the electronic apparatus 100 may input the captured image acquired by the camera 190 to the nutritional ingredient module 10 as the food information.

FIG. 5 is a view illustrating an operation of acquiring nutritional ingredient information according to an embodiment of the disclosure.

Referring to an embodiment 500 of FIG. 5, the electronic apparatus 100 may acquire the nutritional ingredient information corresponding to the food information through the nutritional ingredient module 10. The electronic apparatus 100 may use the nutritional ingredient database 11 to acquire the nutritional ingredient information corresponding to the food information.

The nutritional ingredient information may include information related to various types of nutritional ingredients that correspond to the specific food. The nutritional ingredient information may include the specific nutritional ingredient and the capacity value corresponding to the specific nutritional ingredient.

According to an embodiment of the disclosure, the nutritional ingredient may include at least one of energy (calories), protein, carbohydrates, sugar, dietary fiber, fat (or lipid), saturated fatty acid, monounsaturated fatty acid, polyunsaturated fatty acid, cholesterol, vitamins, minerals, or water. The vitamins may be classified by type, such as vitamins A, C, D, E, and K.

The capacity value may include the mass value. A unit of the mass value may be different based on the nutritional ingredient. The unit of the mass value may include at least one of mg, g, or kg.

Referring to an embodiment 510 of FIG. 5, the nutritional ingredient module 10 may analyze the nutritional ingredient information in various forms. The embodiment 500 shows 11 types of nutritional ingredients, and the embodiment 510 shows 16 types of nutritional ingredients. The type and number of the nutritional ingredients may be changed based on the user setting.

FIG. 6 is a view illustrating an operation of acquiring probability information of a candidate food according to an embodiment of the disclosure.

Referring to an embodiment 600 of FIG. 6, the electronic apparatus 100 may acquire the candidate food probability information corresponding to the nutritional ingredient information through the candidate food probability module 20. The candidate food probability information corresponding to the nutritional ingredient information may be described as the candidate food probability information corresponding to the food information.

The candidate food may include at least one of salad, dumplings, pasta, sandwich, or others. The candidate food may include the predetermined food for which the analysis accuracy is determined not to be high based on the food information. The candidate food may be changed based on the user setting.

FIG. 7 is a view illustrating an operation of acquiring candidate food probability information by using an artificial intelligence model according to an embodiment of the disclosure.

Referring to an embodiment 700 of FIG. 7, the candidate food probability module 20 may receive the nutritional ingredient information as the input data. The candidate food probability module 20 may acquire the candidate food probability information as the output data based on the nutritional ingredient information.

The candidate food probability information may include the probability value corresponding to each candidate food.

The candidate food probability module 20 may be a module included in the artificial intelligence model. The candidate food probability module 20 may generate the candidate food probability information by using the input layer, the hidden layer, and the output layer.

FIG. 8 is a view illustrating an operation of acquiring food group probability information according to an embodiment of the disclosure.

Referring to an embodiment 800 of FIG. 8, the electronic apparatus 100 may acquire the food group probability information through the food group probability module 30. The electronic apparatus 100 may acquire the food group probability information based on at least one of the nutritional ingredient information or the candidate food probability information. The food group probability information may include the probability value indicating to which food group the food information corresponds.

The food group may include at least one of the dairy products, the beans-cereals, the green vegetables, the refined grains, the plant-based proteins, the seafood proteins, the fruit juices, the meat-poultry-egg proteins, other vegetables, the starchy vegetables, the fruits, or the grains. The food group may be changed based on the user setting.

FIG. 9 is a view illustrating an operation of acquiring food group probability information by using an artificial intelligence model according to an embodiment of the disclosure.

Referring to an embodiment 900 of FIG. 9, the food group probability module 30 may receive each of the nutritional ingredient information and the candidate food probability information as the input data. The food group probability module 30 may acquire the food group probability information as the output data based on the nutritional ingredient information and the candidate food probability information.

The food group probability module 30 may be a module included in the artificial intelligence model. The food group probability module 30 may generate the food group probability information by using the input layer, the hidden layer, and the output layer.

FIG. 10 is a view illustrating an operation of acquiring preliminary food group capacity information according to an embodiment of the disclosure.

Referring to an embodiment 1000 of FIG. 10, the electronic apparatus 100 may acquire the preliminary food group capacity information by using the food group capacity module 50. The electronic apparatus 100 may acquire the preliminary food group capacity information corresponding to the food information based on at least one of the nutritional ingredient information or the candidate food probability information. The electronic apparatus 100 may acquire the preliminary food group capacity information based on the food group capacity database in performing the learning operation.

The preliminary food group capacity information may include the capacity corresponding to each food group. The preliminary food group capacity information may include at least one of capacities of the dairy products, the beans-cereals, the green vegetables, the refined grains, the vegetable proteins, the seafood proteins, the fruit juices, the meat-poultry-egg proteins, other vegetables, the starchy vegetables, the fruits, or the grains.

The capacity may be expressed based on the unit capacity. The unit capacity may be at least one of g, mg, kg, 1, ml, cup, or oz.

FIG. 11 is a view illustrating an operation of acquiring preliminary food group capacity information by using an artificial intelligence model according to an embodiment of the disclosure.

Referring to an embodiment 1100 of FIG. 11, the food group capacity module 50 may acquire, as the input data, the nutritional ingredient information and the candidate food probability information. The food group capacity module 50 may acquire the preliminary food group capacity information as the output data based on the nutritional ingredient information and the candidate food probability information.

The food group capacity module 50 may be a module included in the artificial intelligence model. The food group capacity module 50 may generate the preliminary food group capacity information by using the input layer, the hidden layer, and the output layer.

FIG. 12 is a view illustrating a filtering operation of probability information according to an embodiment of the disclosure.

Referring to an embodiment 1200 of FIG. 12, the electronic apparatus 100 may filter the food group probability information by using the filtering module 40. The electronic apparatus 100 may perform the filtering function by using the food group probability information and the food group threshold value information.

The filtering module 40 may acquire the result information by filtering the food group probability information. The result information may be described as the filtered food group probability information.

The food group threshold value information may include the threshold value corresponding to each food group. For example, the food group threshold value information may include a first threshold value of the first food group, a second threshold value of the second food group, or the like.

According to various embodiments of the disclosure, the first threshold value and the second threshold value may be values different from each other.

According to various embodiments of the disclosure, the first threshold value and the second threshold value may be the same value.

According to various embodiments of the disclosure, the threshold values corresponding to all the food groups may be the same.

FIG. 13 is a view illustrating an operation of acquiring final food group capacity information according to an embodiment of the disclosure.

Referring to an embodiment 1300 of FIG. 13, the electronic apparatus 100 may acquire the final food group capacity information by using the final food group capacity module 60. The electronic apparatus 100 may acquire the final food group capacity information based on at least one of the food group probability information or the preliminary food group capacity information.

The electronic apparatus 100 may determine to which food group the food information corresponds based on the food group probability information, and to determine what capacity of which food group the food information corresponds to by using the preliminary food group capacity information. The food group classification operation of the electronic apparatus 100 may have high accuracy in that the electronic apparatus 100 determines the food group based on the two information.

The electronic apparatus 100 may generate, as the final output data, only information including a significant value (e.g., a value other than zero) in the final food group capacity information.

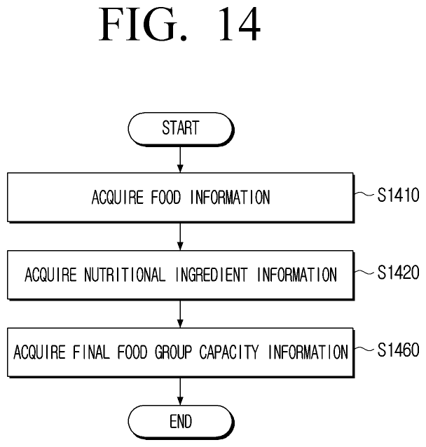

FIG. 14 is a flowchart illustrating an operation of acquiring final food group capacity information according to an embodiment of the disclosure.

Referring to FIG. 14, the electronic apparatus 100 may acquire the food information at operation S1410. The user input for the food may include at least one of the text information, the image information, or the audio information, indicating the food.

The electronic apparatus 100 may acquire the nutritional ingredient information at operation S1420. The electronic apparatus 100 may acquire the nutritional ingredient information based on the food information. The nutritional ingredient information may include at least one nutritional ingredient corresponding to the food information. The nutritional ingredient information may include the capacity information. The nutritional ingredient information may include the capacity of the specific nutritional ingredient.

The electronic apparatus 100 may acquire the final food group capacity information at operation S1460. The electronic apparatus 100 may acquire the final food group capacity information based on at least one of the food information or the nutritional ingredient information. The final food group capacity information may include the capacity corresponding to the specific food group.

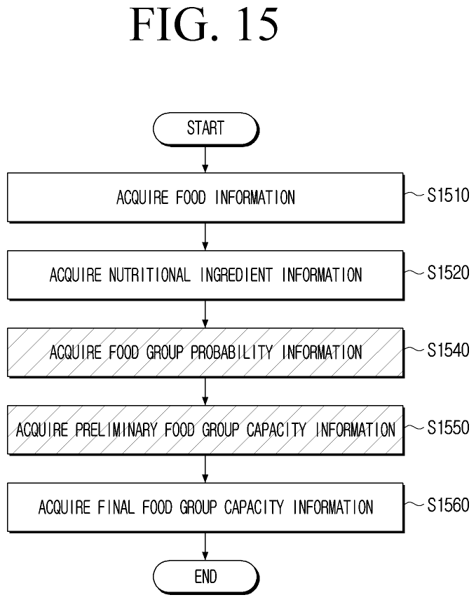

FIG. 15 is a flowchart illustrating an operation of acquiring final food group capacity information by using food group probability information and preliminary food group capacity information according to an embodiment of the disclosure.

Referring to FIG. 15, operations S1510, S1520, and S1560 may correspond to the operations S1410, S1420, and S1460 of FIG. 14. Therefore, their redundant descriptions are omitted.

The electronic apparatus 100 may acquire the food group probability information after acquiring the nutritional ingredient information at operation S1540. The electronic apparatus 100 may acquire the food group probability information based on at least one of the food information or the nutritional ingredient information. The food group probability information may include the probability value indicating to which food the food information corresponds.

The electronic apparatus 100 may acquire the preliminary food group capacity information at operation S1550. The electronic apparatus 100 may acquire the preliminary food group capacity information based on at least one of the food information or the nutritional ingredient information. The preliminary food group capacity information may be information indicating what capacity of which food group the food information corresponds to.

The electronic apparatus 100 may acquire the final food group capacity information based on at least one of the food information, the nutritional ingredient information, the food group probability information, or the preliminary food group capacity information at operation S1560.

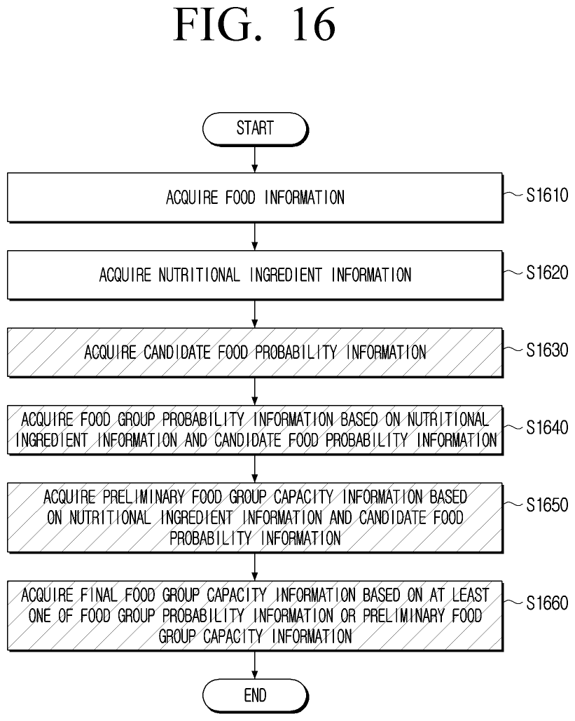

FIG. 16 is a flowchart illustrating an operation of acquiring candidate food probability information according to an embodiment of the disclosure.

Referring to FIG. 16, operations S1610, S1630, S1640, S1650, and S1660 may correspond to the operations S1510, S1540, S1550, and S1560 of FIG. 15. Therefore, their redundant descriptions are omitted.

The electronic apparatus 100 may acquire the candidate food probability information after acquiring the nutritional ingredient information at operation S1630. The candidate food probability information may include the probability value that the food information corresponds to the predetermined food. The predetermined food may include a food for which the analysis accuracy related to the food group is relatively low.

The electronic apparatus 100 may acquire the food group probability information based on the nutritional ingredient information and the candidate food probability information at operation S1640. Unlike FIG. 15, the electronic apparatus 100 may further use the candidate food probability information in acquiring the food group probability information.

The electronic apparatus 100 may acquire the preliminary food group capacity information based on the nutritional ingredient information and the candidate food probability information at operation S1650. Unlike FIG. 15, the electronic apparatus 100 may further use the candidate food probability information in acquiring the preliminary food group capacity information.

The electronic apparatus 100 may acquire the final food group capacity information based on at least one of the food group probability information or the preliminary food group capacity information at operation S1660.

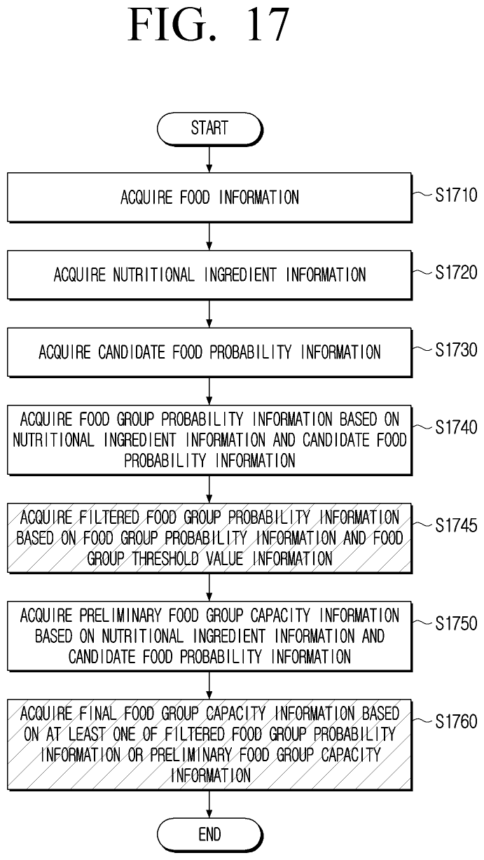

FIG. 17 is a flowchart illustrating a filtering operation of food group probability information according to an embodiment of the disclosure.

Referring to FIG. 17, operations S1710, S1720, S1730, S1740, S1750, and S1760 may correspond to the operations S1610, S1620, S1630, S1640, S1650, and S1660. Therefore, their redundant descriptions are omitted

The electronic apparatus 100 may acquire the filtered food group probability information based on the food group probability information and the food group threshold value information after acquiring the food group probability information at operation S1745.

According to various embodiments of the disclosure, the electronic apparatus 100 may perform the filtering operation based on one representative threshold value. The electronic apparatus 100 may remove (or filter) the information having a probability value less than the representative threshold value from the food group probability information.

According to various embodiments of the disclosure, the electronic apparatus 100 may perform the filtering operation based on the plurality of threshold values. A different threshold value may correspond to each food group. The electronic apparatus 100 may remove (or filter) the information having the probability value less than the threshold value from the food group probability information by comparing the threshold value for each food group in the food group probability information.

The electronic apparatus 100 may acquire the final food group capacity information based on at least one of the filtered food group probability information or the preliminary food group capacity information at operation S1760.

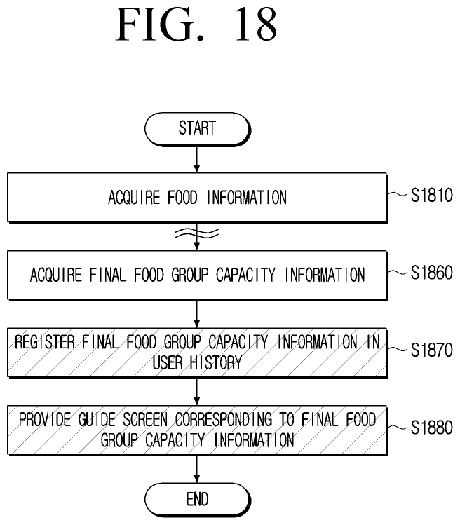

FIG. 18 is a flowchart illustrating an operation of providing a guide screen according to an embodiment of the disclosure.

Referring to FIG. 18, operations S1810 to S1860 may correspond to the operations S1710 to S1760 of FIG. 17. Therefore, their redundant descriptions are omitted.

The electronic apparatus 100 may acquire the final food group capacity information corresponding to the food information. The electronic apparatus 100 may register the final food group capacity information in the user history after acquiring the final food group capacity information at operation S1870. The user history may include various information related to the user eating habit.

The user history may be information storing data on the food information input by the user for the predetermined time period. The predetermined time period may be changed based on the user setting.

The electronic apparatus 100 may provide the guide screen corresponding to the final food group capacity information at operation S1880. The guide screen may include the analysis result of the food information. The analysis result may include the final food group capacity information and the guide information acquired based on the final food group capacity information.

The guide information may include information recommending the specific food to the user.

FIG. 19 is a view illustrating a screen for inputting food information according to an embodiment of the disclosure.

Referring to FIG. 19, a screen 1900 may be a screen corresponding to an eating habit application provided by the electronic apparatus 100.

The screen 1900 may include at least one of text information 1910 guiding an input of the food information or user interface (UI) information 1920 for inputting the food information.

For example, the text information 1910 may include “Please enter the food you consumed today.”

For example, the UI information 1920 may include at least one of the information on the time (e.g., breakfast, lunch, or dinner) at which the user consumes the food, the food (e.g., yogurt, hamburger, or pizza), or the capacity (e.g., 100 ml, 200 mg, or 400 mg).

The electronic apparatus 100 may receive content included in the UI information 1920 directly from the user.

According to various embodiments of the disclosure, the electronic apparatus 100 may acquire the food information based on a recent history. The electronic apparatus 100 may acquire the food information based on the recent history without direct data in case of receiving a command to call the recent history from the user.

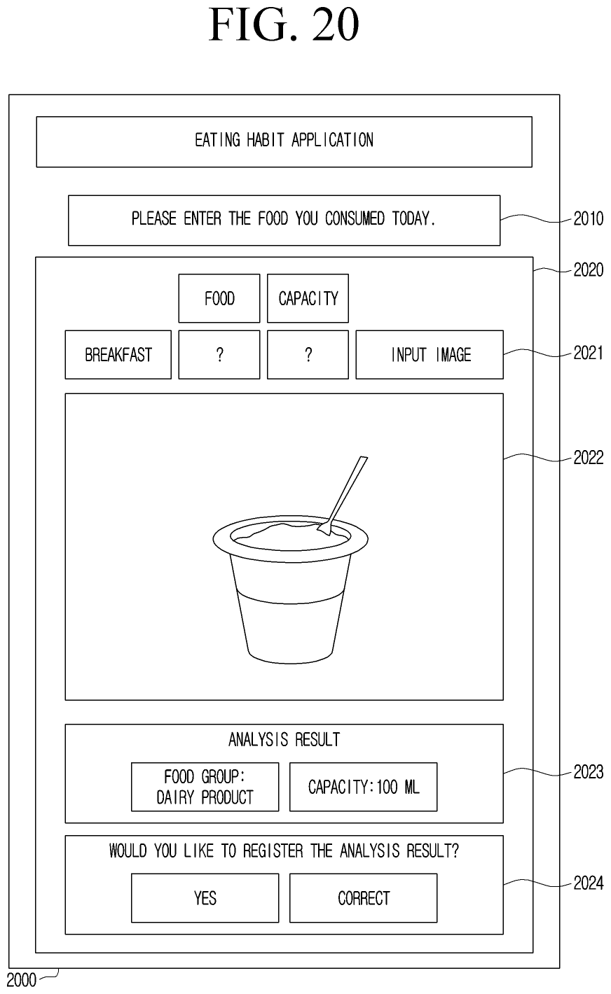

FIG. 20 is a view illustrating a screen for inputting food information by using an image according to an embodiment of the disclosure.

Referring to FIG. 20, a screen 2000 may be a screen corresponding to the eating habit application provided by the electronic apparatus 100.

The screen 2000 may include text information 2010 guiding the input of the food information, UI information 2020 for inputting the food information, UI information 2023 indicating the analysis result, and UI information 2024 for registering the analysis result.

The text information 2010 may correspond to the text information 1910 of FIG. 19. Therefore, its redundant description is omitted.

The UI information 2020 for inputting the food information may include at least one of a UI 2021 guiding the user command for inputting the food information as an image or a UI 2022 including an image to be used as the food information. The image may be an image pre-stored in the electronic apparatus 100.

According to various embodiments of the disclosure, the image may be an image captured using the eating habit application.

The UI information 2023 indicating the analysis result may include a result of analyzing the food information. For example, the UI information 2023 may include the food group (e.g., the dairy product) and the capacity (100 ml) corresponding to yogurt in case that the food information is yogurt.

The UI information 2024 for registering the analysis result may include information for registering (or storing) the analysis result provided through the UI information 2023 in the user history. The electronic apparatus 100 may register (or store) the analysis result in the user history in case of receiving the user input through the UI information 2024.

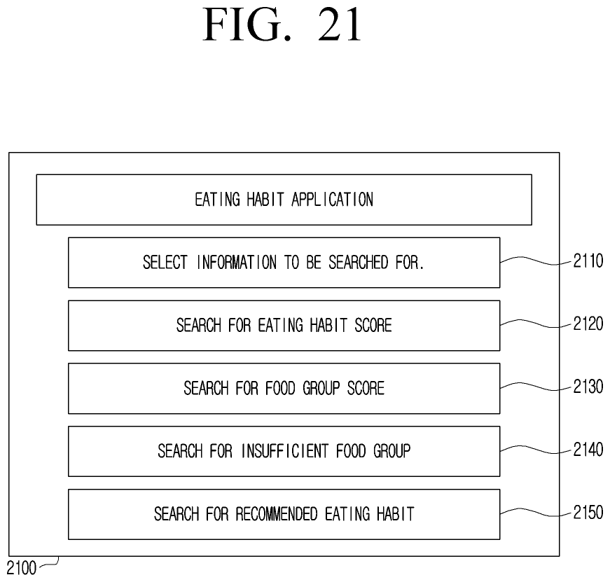

FIG. 21 is a view illustrating a guide screen related to a user history according to an embodiment of the disclosure.

Referring to FIG. 21, a screen 2100 may be a screen corresponding to the eating habit application provided by the electronic apparatus 100.

The screen 2100 may include at least one of a UI 2110 guiding selection of information to be searched for, a UI 2120 guiding the search for the eating habit score, a UI 2130 guiding the search for the food group score, a UI 2140 guiding the search for the insufficient food group, or a UI 2150 guiding the search for the recommended eating habit.

The electronic apparatus 100 may provide a screen corresponding to the user input in case of receiving an input for selecting a specific UI from the user. Screens related thereto are described with reference to FIGS. 22 to 25.

FIG. 22 is a view illustrating a screen related to an eating habit score according to an embodiment of the disclosure.

Referring to FIG. 22, a screen 2200 may be a screen corresponding to the eating habit application provided by the electronic apparatus 100.

The screen 2200 may include at least one of a UI 2210 indicating the eating habit score, a UI 2220 indicating the user age, a UI 2230 indicating the average score of a user age group, a UI 2240 indicating an evaluation result, or a UI 2250 indicating an expected eating habit score.

The electronic apparatus 100 may calculate the eating habit score based on the user history. The electronic apparatus 100 may update the user history based on the final food group capacity information and calculate the eating habit score based on the updated user history. The electronic apparatus 100 may provide the eating habit score through the UI 2210.

The electronic apparatus 100 may provide the user age included in the user history through the UI 2220.

The electronic apparatus 100 may provide an average eating habit score of another user similar to the user age through the UI 2230.

The electronic apparatus 100 may provide the evaluation result related to the user eating habit (or eating habit score) through the UI 2240 based on the user history.

The electronic apparatus 100 may assume that the user maintains a current eating habit, and provide the eating habit score which is highly likely to be calculated after the predetermined time period (e.g., 1 week or 1 month) through the UI 2250.

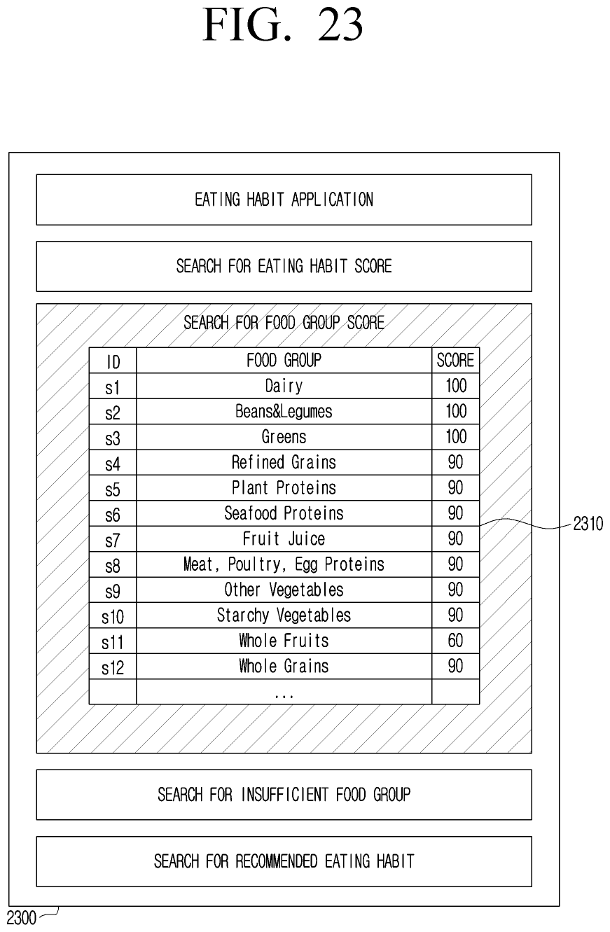

FIG. 23 is a view illustrating a screen related to a food group score according to an embodiment of the disclosure.

Referring to FIG. 23, a screen 2300 may be a screen corresponding to the eating habit application provided by the electronic apparatus 100.

The screen 2300 may include a UI 2310 indicating food group score information. The food group score may be a score indicating whether the user appropriately consumes each food group.

The electronic apparatus 100 may acquire the food group score information including the score of each food group based on the user history. The food group score information may include a user score corresponding to each predetermined food group.

The electronic apparatus 100 may provide the food group score information through the UI 2310. The user may easily determine whether the user appropriately consumes the food group through the UI 2310.

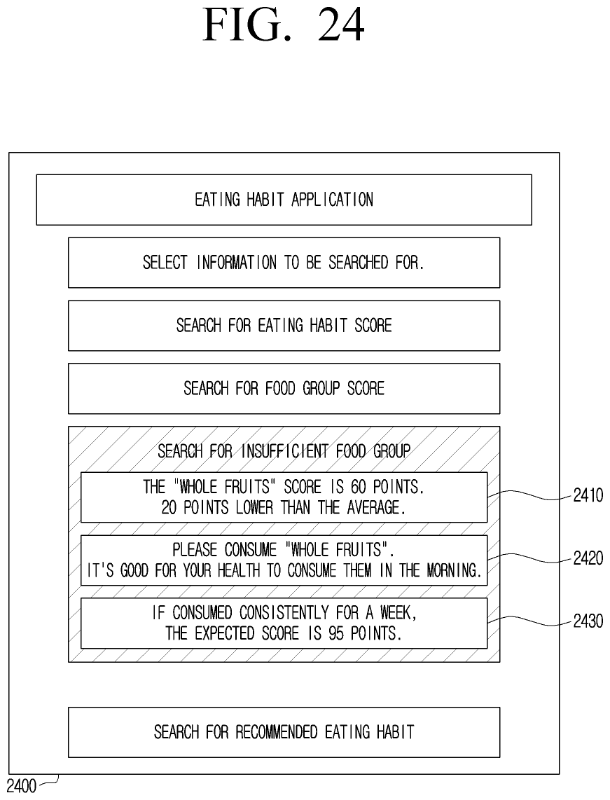

FIG. 24 is a view illustrating a screen related to an insufficient food group according to an embodiment of the disclosure.

Referring to FIG. 24, a screen 2400 may be a screen corresponding to the eating habit application provided by the electronic apparatus 100.

The screen 2400 may include at least one of a UI 2410 indicating the evaluation result related to the insufficient food group, a UI 2420 guiding the user consumption of the insufficient food group, or a UI 2430 indicating the expected score calculated in case that the user consumes the insufficient food group.

The electronic apparatus 100 may acquire the food group score information based on the user history. The electronic apparatus 100 may identify the insufficient food group based on the user history. The electronic apparatus 100 may identify a food group having a score of the threshold value or less as the insufficient food group based on the food group score information.

The electronic apparatus 100 may provide various information based on the insufficient food group. The electronic apparatus 100 may provide at least one of a score corresponding to the insufficient food group or an average score corresponding to the insufficient food group through the UI 2410.

The electronic apparatus 100 may provide information recommending the user consumption of the insufficient food group through the UI 2420.

The electronic apparatus 100 may calculate the expected improved score in case that the user consumes the insufficient food group, and provide the calculated expected score through the UI 2430.

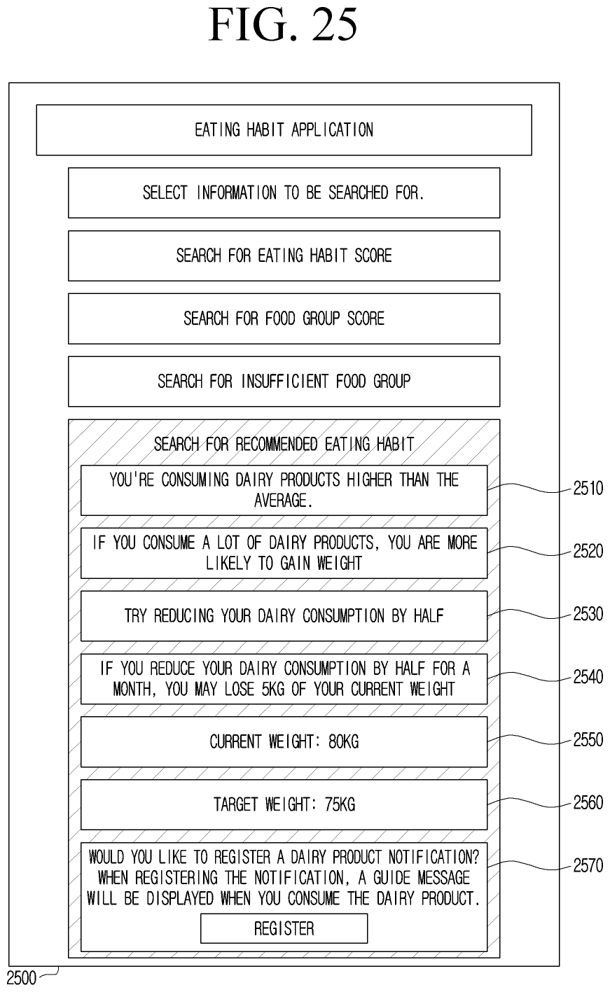

FIG. 25 is a view illustrating a screen related to a recommended eating habit according to an embodiment of the disclosure.

Referring to FIG. 25, a screen 2500 may be a screen corresponding to the eating habit application provided by the electronic apparatus 100.

The screen 2500 may include at least one of UIs 2510, 2520, and 2530 each guiding the recommended eating habit, a UI 2540 indicating an effect of the recommended eating habit, a UI 2550 indicating a user weight, a UI 2560 indicating a target weight, or a UI 2570 guiding registration of a notification related to the recommended eating habit.

The electronic apparatus 100 may determine the recommended eating habit based on the user history. The electronic apparatus 100 may identify the excessively consumed food group. The electronic apparatus 100 may provide the guide information related to the excessively consumed food group.

The electronic apparatus 100 may provide information indicating the user evaluation result related to the excessively consumed food group through the UI 2510.

The electronic apparatus 100 may provide information indicating a side effect related to the excessively consumed food group through the UI 2520.

The electronic apparatus 100 may provide information indicating the recommended capacity of the excessively consumed food group through the UI 2530.

The electronic apparatus 100 may provide information indicating the effect of the recommended eating habit through the UI 2540. The electronic apparatus 100 may provide information indicating a user current weight stored in the user history through the UI 2550. The electronic apparatus 100 may provide a weight that the user may achieve through the recommended eating habit or a standard weight suitable for the user through the UI 2560.

The electronic apparatus 100 may provide the guide information for registering the notification related to the excessively consumed food group through the UI 2570. The electronic apparatus 100 may warn the user about the excessively consumed food group. The electronic apparatus 100 may provide a caution notification to warn about the excessively consumed food group.

The electronic apparatus 100 may provide a warning notification to the user. The electronic apparatus 100 may provide the warning notification related to the excessively consumed food group in case of receiving the user input through the UI 2570. The electronic apparatus 100 may provide the warning notification based on a predetermined event. The electronic apparatus 100 may provide the warning notification to the user in case of recognizing the excessively consumed food group through the final food group capacity information.

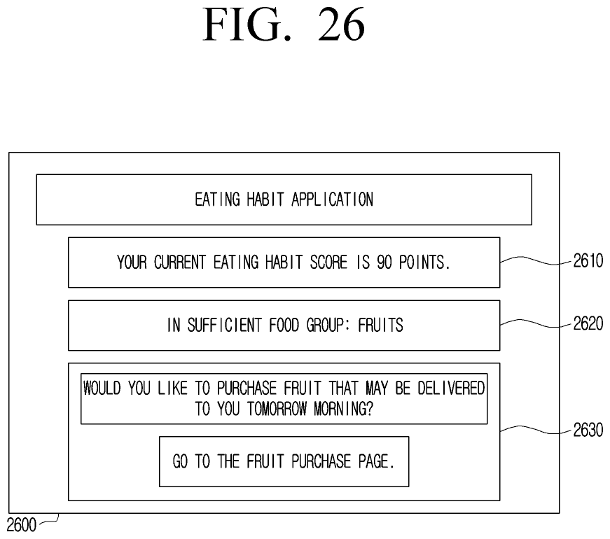

FIG. 26 is a view illustrating a screen providing guide information for purchase of an insufficient food group according to an embodiment of the disclosure.

Referring to FIG. 26, a screen 2600 may be a screen corresponding to the eating habit application provided by the electronic apparatus 100.

The screen 2600 may include at least one of a UI 2610 indicating the eating habit score, a UI 2620 indicating the insufficient food group, or a UI 2630 indicating information related to the purchase of the insufficient food group.

The electronic apparatus 100 may acquire the user eating habit score based on the user history. The electronic apparatus 100 may provide the eating habit score through the UI 2610.

The electronic apparatus 100 may identify the insufficient food group based on the user history and provide the identified insufficient food group through the UI 2620.

The electronic apparatus 100 may provide a method for purchasing the insufficient food group through the UI 2630. The electronic apparatus 100 may provide the UI 2630 guiding online address information where the insufficient food group may be purchased. The electronic apparatus 100 may provide address information of an internet site where the insufficient food group may be purchased.

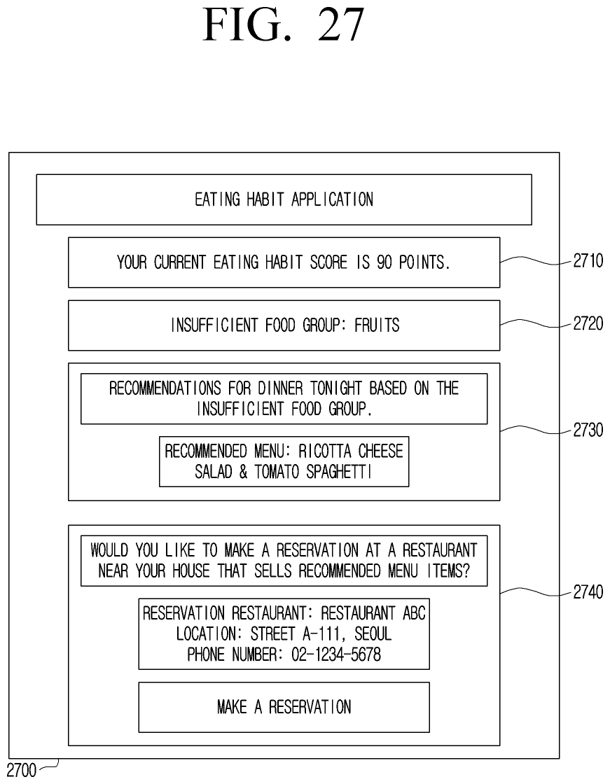

FIG. 27 is a view illustrating a screen providing restaurant information related to an insufficient food group according to an embodiment of the disclosure.

Referring to FIG. 27, a screen 2700 may be a screen corresponding to the eating habit application provided by the electronic apparatus 100.

The screen 2700 may include at least one of a UI 2710 indicating the eating habit score, a UI 2720 indicating the insufficient food group, a UI 2730 indicating a recommended food for the insufficient food group, or a UI 2740 indicating place information related to the recommended food.

The UIs 2710 and 2720 may correspond to the UIs 2610 and 2620 of FIG. 26. Therefore, their redundant descriptions are omitted.

The electronic apparatus 100 may determine the recommended food related to the insufficient food group among the plurality of pre-stored food. The recommended food may be a food that may supplement the nutritional ingredient for the insufficient food group.

The electronic apparatus 100 may provide information related to recommended food through the UI 2730.

The electronic apparatus 100 may provide a reservation service related to the recommended food through the UI 2740. The UI 2740 may include text information guiding a reservation related to the recommended food. The UI 2740 may include the name, location, phone number, or the like of the restaurant related to the recommended food. The electronic apparatus 100 may receive the user input for making a restaurant reservation through the UI 2740.

FIG. 28 is a flowchart illustrating an operation of acquiring a final food group capacity information by using a server according to an embodiment of the disclosure.

Referring to FIG. 28, operations S2810, S2820, S2830, S2840, S2845, S2850, and S2860 may correspond to the operations S1710, S1720, S1730, S1740, S1745, S1750, and S1760 of FIG. 17. Therefore, their redundant descriptions are omitted.

The electronic apparatus 100 may transmit the food information to the server 200 after acquiring the food information at operation S2811.

The server 200 may receive the food information from the electronic apparatus 100. The server 200 may acquire the final food group capacity information based on the received food information.

The server 200 may acquire the nutritional ingredient information based on the food information at operation S2820.

The server 200 may acquire the candidate food probability information based on the nutritional ingredient information at operation S2830.

The server 200 may acquire the food group probability information based on the nutritional ingredient information and the candidate food probability information at operation S2840.

The server 200 may acquire the filtered food group probability information based on the food group probability information and the food group threshold value information at operation S2845.

The server 200 may acquire the preliminary food group capacity information based on the nutritional ingredient information and the candidate food probability information at operation S2850.

The server 200 may acquire the final food group capacity information based on the filtered food group probability information and the preliminary food group capacity information at operation S2860.

The server 200 may register the final food group capacity information in the user history. According to various embodiments of the disclosure, a registration operation may be performed by the electronic apparatus 100.

The server 200 may transmit the final food group capacity information to the electronic apparatus 100 at operation S2861.

The electronic apparatus 100 may receive the final food group capacity information from the server 200. The electronic apparatus 100 may provide a guide screen corresponding to the final food group capacity information at operation S2880.

FIG. 29 is a flowchart illustrating an operation of generating guide information by using a server according to an embodiment of the disclosure.

Referring to FIG. 29, operations S2910, S2911, S2920, S2930, S2940, S2945, S2950, and S2960 may correspond to the operations S2810, S2811, S2820, S2830, S2840, S2845, S2850, and S2860 of FIG. 28.

The server 200 may register the final food group capacity information in the user history after acquiring the final food group capacity information at operation S2970. The server 200 may generate the guide information based on the user history at operation S2975. The server 200 may transmit the guide information to the electronic apparatus 100 at operation S2976.

The electronic apparatus 100 may receive the guide information from the server 200. The electronic apparatus 100 may provide a guide screen including the received guide information at operation S2980.

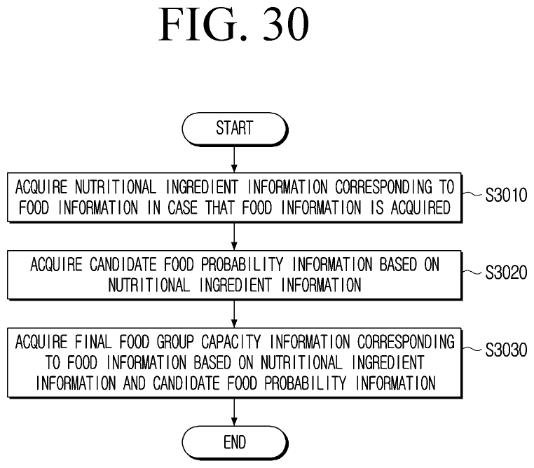

FIG. 30 is a flowchart illustrating a method of controlling an electronic apparatus according to an embodiment of the disclosure.

Referring to FIG. 30, the method of an electronic apparatus 100 may include acquiring nutritional ingredient information corresponding to food information in case that the food information (or information including a user input for a food) is acquired at operation S3010, acquiring candidate food probability information based on the nutritional ingredient information at operation S3020, and acquiring final food group capacity information corresponding to the food information based on the nutritional ingredient information and the candidate food probability information at operation S3030.

In the acquiring of the final food group capacity information, food group probability information may be acquired based on the nutritional ingredient information and the candidate food probability information, and the final food group capacity information may be acquired based on the food group probability information.

In the acquiring of the final food group capacity information, filtering may be performed for the food group probability information by removing a probability value less than a threshold value from the food group probability information, the filtered food group probability information may be acquired as a filtering operation result, and the final food group capacity information may be acquired based on the filtered food group probability information.

In the performing of the filtering, the filtering operation may be performed based on food group threshold value information including the threshold value corresponding to each of the plurality of food groups.

In the acquiring of the final food group capacity information, preliminary food group capacity information may be acquired based on the nutritional ingredient information and the candidate food probability information, and the final food group capacity information may be acquired based on the preliminary food group capacity information.

The user input for the food may include at least one of text information, image information, or audio information, indicating a specific food.

The method of controlling an electronic device may further include updating a user history indicating a user eating habit based on the final food group capacity information.

The method of controlling an electronic device may further include acquiring score information related to the user eating habit based on the updated user history.

The method of controlling an electronic device may further include providing guide information indicating a recommended eating habit based on the updated user history, and the guide information may include at least one of information related to an insufficient food group or information related to an excessively consumed food group.

The method of controlling an electronic device may further include displaying a guide screen including the food information and the final food group capacity information.

Meanwhile, the methods according to the various embodiments of the disclosure described above may be implemented in the form of an application which may be installed on an electronic apparatus of the related art.

In addition, the methods according to the various embodiments of the disclosure described above may be implemented only by software upgrade or hardware upgrade of the electronic apparatus of the related art.

In addition, the various embodiments of the disclosure described above may be performed through an embedded server included in the electronic apparatus, or an external server of at least one of the electronic apparatus or a display device.

Meanwhile, according to an embodiment of the disclosure, the various embodiments described above may be implemented by software including an instruction stored in a machine-readable storage medium (for example, a computer-readable storage medium). A machine may be a device that invokes the stored instruction from a storage medium, may be operated based on the invoked instruction, and may include the electronic apparatus in the disclosed embodiments. In case that the instruction is executed by the processor, the processor may perform a function corresponding to the instruction directly or by using other components under control of the processor. The instruction may include codes generated or executed by a compiler or an interpreter. The machine-readable storage medium may be provided in the form of a non-transitory storage medium. Here, the term “non-transitory” indicates that the storage medium is tangible without including a signal, and does not distinguish whether data are semi-permanently or temporarily stored in the storage medium.

In addition, according to an embodiment of the disclosure, the methods according to the various embodiments described above may be provided by being included in a computer program product. The computer program product may be traded as a product between a seller and a purchaser. The computer program product may be distributed in a form of the machine-readable storage medium (for example, compact disc read only memory (CD-ROM)), or may be distributed online through an application store (for example, PlayStore™). In case of the online distribution, at least some of the computer program products may be at least temporarily stored in a storage medium, such as memory of a server of a manufacturer, a server of an application store, or a relay server, or be temporarily generated.

In addition, each of the components (for example, modules or programs) according to the various embodiments described above may include a single entity or a plurality of entities, and some of the corresponding sub-components described above may be omitted or other sub-components may be further included in the various embodiments. Alternatively or additionally, some of the components (e.g., modules or programs) may be integrated into one entity, and may perform functions performed by the respective corresponding components before being integrated in the same or similar manner. Operations performed by the modules, the programs, or other components according to the various embodiments may be executed in a sequential manner, a parallel manner, an iterative manner, or a heuristic manner, at least some of the operations may be performed in a different order or be omitted, or other operations may be added.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic apparatus comprising:
memory storing one or more computer programs; and
one or more processors communicatively coupled to the memory,
wherein the one or more computer programs include computer-executable instructions executed by the one or more processors, and
wherein the one or more processors configured to:
receive a user input for a food,
acquire food information corresponding to the food from the received user input,
acquire nutritional ingredient information of the acquired food information by inputting the acquired food information into a nutritional ingredient module,
acquire candidate food probability information by inputting the nutritional ingredient information into a candidate food probability module,
acquire food group probability information and preliminary food group capacity information by inputting the acquired nutritional ingredient information and the acquired candidate food probability information respectively into a food group probability module and a food group capacity module,
acquire filtered food group probability information by removing a probability value less than a threshold value from the acquired food group probability information, and
acquire final food group capacity information by coupling the filtered food group probability information with the preliminary food group capacity information.

2. The apparatus of claim 1, wherein the one or more processors is configured to
acquire food group threshold value information including the threshold value corresponding to each of a plurality of food groups; and
acquire the filtered food group probability information by removing the probability value less than the threshold value included in the food group threshold value information from the acquired food group probability information for each of the plurality of food groups.

3. The apparatus of claim 1, wherein the one or more processors is configured to acquire the preliminary food group capacity information indicating capacity information for each of a plurality of food groups by inputting the nutritional ingredient information and the candidate food probability information into the food group capacity module.

4. The apparatus of claim 1, wherein the user input for the food includes at least one of text information, image information, or audio information, indicating a specific food.

5. The apparatus of claim 1, wherein the one or more processors is configured to
store, in the memory, a user history including accumulated capacity information for each of a plurality of food groups consumed by a user during a predetermined time period, and
update the user history by accumulating a capacity value for each of the plurality of food groups that is included in the final food group capacity information.

6. The apparatus of claim 5, wherein the one or more processors is configured to acquire score information related to a user eating habit by comparing reference capacity information with the accumulated capacity information for each of a plurality of food groups that is included in the updated user history.

7. The apparatus of claim 6, wherein the score information includes at least one of a user eating habit score, a user age, or an average score of the user age.

8. The apparatus of claim 6, wherein the one or more processors is configured to provide guide information indicating a recommended eating habit for a first food group by comparing a first capacity value of the first food group that is included in the updated user history with a first reference value corresponding to the first food group that is included in the reference capacity information.

9. The apparatus of claim 8, wherein the guide information includes at least one of information related to an insufficient food group or information related to an excessively consumed food group.

10. The apparatus of claim 1, further comprising:
a display,
wherein the one or more processors is configured to control the display to display a guide screen including the food information and the final food group capacity information.

11. A method of controlling an electronic apparatus, the method comprising:
receiving a user input for a food;
acquiring food information corresponding to the food from the received user input;
acquiring nutritional ingredient information of the acquired food information by inputting the acquired food information into a nutritional ingredient module;
acquiring candidate food probability information by inputting the nutritional ingredient information into a candidate food probability module;
acquiring food group probability information and preliminary food group capacity information by inputting the acquired nutritional ingredient information and the acquired candidate food probability information respectively into a food group probability module and a food group capacity module;
acquiring filtered food group probability information by removing a probability value less than a threshold value from the acquired food group probability information; and
acquiring final food group capacity information by coupling the filtered food group probability information with the preliminary food group capacity information.

12. The method of claim 11, wherein the acquiring of the filtered food group probability information further comprises:
acquiring food group threshold value information including the threshold value corresponding to each of a plurality of food groups is acquired; and
acquiring the filtered food group probability information by removing the probability value less than the threshold value included in the food group threshold value information from the acquired food group probability information for each of the plurality of food groups.

13. The method of claim 11, wherein in the acquiring of the preliminary food group capacity information further comprises acquiring the preliminary food group capacity information indicating capacity information for each of a plurality of food groups by inputting the nutritional ingredient information and the candidate food probability information into the food group capacity module.

14. The method of claim 11, wherein the user input for the food includes at least one of text information, image information, or audio information, indicating a specific food.

15. The method of claim 11, further comprising:
storing a user history including accumulated capacity information for each of a plurality of food groups consumed by a user during a predetermined time period; and
updating the user history by accumulating a capacity value for each of the plurality of food groups that is included in the final food group capacity information.

16. The method of claim 15, further comprising acquiring score information related to a user eating habit by comparing reference capacity information with the accumulated capacity information for each of the plurality of food groups that is included in the updated user history.

17. The method of claim 16, wherein the score information includes at least one of a user eating habit score, a user age, or an average score of the user age.

18. The method of claim 16, further comprising providing guide information indicating a recommended eating habit for a first food group by comparing a first capacity value of the first food group that is included in the updated user history with a first reference value corresponding to the first food group that is included in the reference capacity information.

19. One or more non-transitory computer-readable storage media storing computer-executable instructions that, when executed by one or more processors of an electronic apparatus, cause the electronic apparatus to perform operations, the operations comprising:
receiving a user input for a food;
acquiring food information corresponding to the food from the received user input;
acquiring nutritional ingredient information of the acquired food information by inputting the acquired food information into a nutritional ingredient module;
acquiring candidate food probability information by inputting the nutritional ingredient information into a candidate food probability module;
acquiring food group probability information and preliminary food group capacity information by inputting the acquired nutritional ingredient information and the acquired candidate food probability information respectively into a food group probability module and a food group capacity module;
acquiring filtered food group probability information by removing a probability value less than a threshold value from the acquired food group probability information; and
acquiring final food group capacity information by coupling the filtered food group probability information with the preliminary food group capacity information.

20. The one or more non-transitory computer-readable storage media of claim 19, wherein the acquiring of the filtered food group probability information further comprises:
acquiring food group threshold value information including the threshold value corresponding to each of a plurality of food groups is acquired; and
acquiring the filtered food group probability information by removing the probability value less than the threshold value included in the food group threshold value information from the acquired food group probability information for each of the plurality of food groups.
